SEQUENCE LISTING (51 sequences)

SEQ ID NO: 1 - DNA, 34 bp, synthetic construct
  misc_feature 1..34: note = dsODN oligonucleotide
  misc_feature 1: note = phosphorylated
  source 1..34: mol_type = other DNA
  modified_base 2^3: mod_base = OTHER; note = phosphorothioate bond
  modified_base 33^34: mod_base = OTHER; note = phosphorothioate bond
gtttaattgagttgtcatatgttaataacggtat

SEQ ID NO: 2 - DNA, 34 bp, synthetic construct
  misc_feature 1..34: note = dsODN oligonucleotide
  misc_feature 1: note = phosphorylated
  modified_base 33^34: mod_base = OTHER; note = phosphorothioate bond
  source 1..34: mol_type = other DNA
  modified_base 2^3: mod_base = OTHER; note = phosphorothioate bond
ataccgttattaacatatgacaactcaattaaac

SEQ ID NO: 3 - protein, 1368 aa, Streptococcus pyogenes [sequence omitted]

SEQ ID NO: 4 - protein, 1053 aa, Streptococcus pyogenes
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 5 - DNA, 21 bp, synthetic construct
  misc_feature 1..21: note = GFPsg1 protospacer
  source 1..21: mol_type = other DNA
gggacggcgacgtaaacggcc

SEQ ID NO: 6 - DNA, 21 bp, synthetic construct
  misc_feature 1..21: note = GFPsg2 protospacer
  source 1..21: mol_type = other DNA
gggcgaggagctgttcaccgg

SEQ ID NO: 7 - DNA, 21 bp, synthetic construct
  misc_feature 1..21: note = GFPsg3 protospacer
  source 1..21: mol_type = other DNA
gcaacatcctggggcacaagc

SEQ ID NO: 8 - DNA, 21 bp, synthetic construct
  misc_feature 1..21: note = GFPsg4 protospacer
  source 1..21: mol_type = other DNA
ggcgtgtccggcgagggcgag

SEQ ID NO: 9 - DNA, 21 bp, synthetic construct
  misc_feature 1..21: note = GFPsg5 protospacer
  source 1..21: mol_type = other DNA
gctcggcgcgggtcttgtagt

SEQ ID NO: 10 - DNA, 21 bp, synthetic construct
  misc_feature 1..21: note = GFPsg6 protospacer
  source 1..21: mol_type = other DNA
ggaacttcacctcggcgcggg

SEQ ID NO: 11 - DNA, 21 bp, synthetic construct
  misc_feature 1..21: note = GFPsg7 protospacer
  source 1..21: mol_type = other DNA
gcacggggccgtcgccgatgg

SEQ ID NO: 12 - DNA, 20 bp, synthetic construct
  misc_feature 1..20: note = GFPsg8 protospacer
  source 1..20: mol_type = other DNA
cacctacggcaagctgaccc

SEQ ID NO: 13 - DNA, 22 bp, synthetic construct
  misc_feature 1..22: note = EMX1_sg1 protospacer
  source 1..22: mol_type = other DNA
gtgtggttccagaaccggagga

SEQ ID NO: 14 - DNA, 21 bp, synthetic construct
  misc_feature 1..21: note = EMX1_sg4 protospacer
  source 1..21: mol_type = other DNA
gctcagcctgagtgttgaggc

SEQ ID NO: 15 - DNA, 21 bp, synthetic construct
  misc_feature 1..21: note = EMX1_sg6 protospacer
  source 1..21: mol_type = other DNA
gcaaccacaaacccacgaggg

SEQ ID NO: 16 - DNA, 21 bp, synthetic construct
  misc_feature 1..21: note = EMX1_sg10 protospacer
  source 1..21: mol_type = other DNA
ggctctccgaggagaaggcca

SEQ ID NO: 17 - DNA, 21 bp, synthetic construct
  misc_feature 1..21: note = EMX1_sg2 protospacer
  source 1..21: mol_type = other DNA
tggccaggctttggggaggcc

SEQ ID NO: 18 - DNA, 20 bp, synthetic construct
  misc_feature 1..20: note = EMX1_sg7 protospacer
  source 1..20: mol_type = other DNA
ggccaggctttggggaggcc

SEQ ID NO: 19 - DNA, 21 bp, synthetic construct
  misc_feature 1..21: note = VEGFA_sg8 protospacer
  source 1..21: mol_type = other DNA
gggtgagtgagtgtgtgcgtg

SEQ ID NO: 20 - DNA, 20 bp, synthetic construct
  misc_feature 1..20: note = AAVS1_sg4 protospacer
  source 1..20: mol_type = other DNA
gactaggaaggaggaggcct

SEQ ID NO: 21 - DNA, 21 bp, synthetic construct
  misc_feature 1..21: note = CCR5_sg2 protospacer
  source 1..21: mol_type = other DNA
gttgccctaaggattaaatga

SEQ ID NO: 22 - DNA, 20 bp, synthetic construct
  misc_feature 1..20: note = EMX1_sg1 forward primer
  source 1..20: mol_type = other DNA
ggagcagctggtcagagggg

SEQ ID NO: 23 - DNA, 22 bp, synthetic construct
  misc_feature 1..22: note = EMX1_sg1 reverse primer
  source 1..22: mol_type = other DNA
ccatagggaagggggacactgg

SEQ ID NO: 24 - DNA, 20 bp, synthetic construct
  misc_feature 1..20: note = EMX1_sg4 forward primer
  source 1..20: mol_type = other DNA
ggagcagctggtcagagggg

SEQ ID NO: 25 - DNA, 22 bp, synthetic construct
  misc_feature 1..22: note = EMX1_sg4 reverse primer
  source 1..22: mol_type = other DNA
ccatagggaagggggacactgg

SEQ ID NO: 26 - DNA, 20 bp, synthetic construct
  misc_feature 1..20: note = EMX1_sg6 forward primer
  source 1..20: mol_type = other DNA
ggagcagctggtcagagggg

SEQ ID NO: 27 - DNA, 22 bp, synthetic construct
  misc_feature 1..22: note = EMX1_sg6 reverse primer
  source 1..22: mol_type = other DNA
ccatagggaagggggacactgg

SEQ ID NO: 28 - DNA, 20 bp, synthetic construct
  misc_feature 1..20: note = EMX1_sg10 forward primer
  source 1..20: mol_type = other DNA
ggagcagctggtcagagggg

SEQ ID NO: 29 - DNA, 22 bp, synthetic construct
  misc_feature 1..22: note = EMX1_sg10 reverse primer
  source 1..22: mol_type = other DNA
ccatagggaagggggacactgg

SEQ ID NO: 30 - DNA, 20 bp, synthetic construct
  misc_feature 1..20: note = EMX1_sg2 forward primer
  source 1..20: mol_type = other DNA
ggagcagctggtcagagggg

SEQ ID NO: 31 - DNA, 22 bp, synthetic construct
  misc_feature 1..22: note = EMX1_sg2 reverse primer
  source 1..22: mol_type = other DNA
ccatagggaagggggacactgg

SEQ ID NO: 32 - DNA, 20 bp, synthetic construct
  misc_feature 1..20: note = EMX1_sg7 forward primer
  source 1..20: mol_type = other DNA
ggagcagctggtcagagggg

SEQ ID NO: 33 - DNA, 22 bp, synthetic construct
  misc_feature 1..22: note = EMX1_sg7 reverse primer
  source 1..22: mol_type = other DNA
ccatagggaagggggacactgg

SEQ ID NO: 34 - DNA, 20 bp, synthetic construct
  misc_feature 1..20: note = VEGFA_sg8 forward primer
  source 1..20: mol_type = other DNA
tccagatggcacattgtcag

SEQ ID NO: 35 - DNA, 20 bp, synthetic construct
  misc_feature 1..20: note = VEGFA_sg8 reverse primer
  source 1..20: mol_type = other DNA
agggagcaggaaagtgaggt

SEQ ID NO: 36 - DNA, 20 bp, synthetic construct
  misc_feature 1..20: note = AAVS1_sg4 forward primer
  source 1..20: mol_type = other DNA
acacctaggacgcaccattc

SEQ ID NO: 37 - DNA, 20 bp, synthetic construct
  misc_feature 1..20: note = AAVS1_sg4 reverse primer
  source 1..20: mol_type = other DNA
cttgctttctttgcctggac

SEQ ID NO: 38 - DNA, 20 bp, synthetic construct
  misc_feature 1..20: note = CCR5_sg2 forward primer
  source 1..20: mol_type = other DNA
ccggccatttcactctgact

SEQ ID NO: 39 - DNA, 20 bp, synthetic construct
  misc_feature 1..20: note = CCR5_sg2 reverse primer
  source 1..20: mol_type = other DNA
ttgctgctagcttccctgtc

SEQ ID NO: 40 - protein, 1053 aa, synthetic construct
  REGION 1..1053: note = KKH SaCas9_N888Q
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLQAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 41 - protein, 1053 aa, synthetic construct
  REGION 1..1053: note = KKH SaCas9_N888Q A889S
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLQSHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 42 - protein, 1053 aa, synthetic construct
  REGION 1..1053: note = KKH SaCas9_N888H A889Q
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLHQHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 43 - protein, 1053 aa, synthetic construct
  REGION 1..1053: note = KKH SaCas9_N888S A889Q
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLSQHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 44 - protein, 1053 aa, synthetic construct
  REGION 1..1053: note = KKH SaCas9_N888R A889Q
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLRQHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 45 - DNA, 3159 bp, synthetic construct
  misc_feature 1..3159: note = nucleotide sequence encoding Streptococcus pyogenes Cas9 Protein with the N888Q mutation
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctatacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccatcgggtggacaagaagcctaatagaaagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgcaggcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaagaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggcccccccacatcattaagacaatcgcctccaagacccagagcattaagaagtacagcacagacattctgggcaacctgtatgaagtgaaatctaagaagcaccctcagatcatcaaaaagggc

SEQ ID NO: 46 - DNA, 3159 bp, synthetic construct
  misc_feature 1..3159: note = nucleotide sequence encoding Streptococcus pyogenes Cas9 Protein with the N888Q and A889S mutations
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctatacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccatcgggtggacaagaagcctaatagaaagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgcagagccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaagaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggcccccccacatcattaagacaatcgcctccaagacccagagcattaagaagtacagcacagacattctgggcaacctgtatgaagtgaaatctaagaagcaccctcagatcatcaaaaagggc

SEQ ID NO: 47 - DNA, 3159 bp, synthetic construct
  misc_feature 1..3159: note = nucleotide sequence encoding Streptococcus pyogenes Cas9 Protein with the N888H and A889Q mutations
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctatacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccatcgggtggacaagaagcctaatagaaagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgcaccagcatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaagaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggcccccccacatcattaagacaatcgcctccaagacccagagcattaagaagtacagcacagacattctgggcaacctgtatgaagtgaaatctaagaagcaccctcagatcatcaaaaagggc

SEQ ID NO: 48 - DNA, 3159 bp, synthetic construct
  misc_feature 1..3159: note = nucleotide sequence encoding Streptococcus pyogenes Cas9 Protein with the N888S and A889Q mutations
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctatacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccatcgggtggacaagaagcctaatagaaagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgagccagcatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaagaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggcccccccacatcattaagacaatcgcctccaagacccagagcattaagaagtacagcacagacattctgggcaacctgtatgaagtgaaatctaagaagcaccctcagatcatcaaaaagggc

SEQ ID NO: 49 - DNA, 3159 bp, synthetic construct
  misc_feature 1..3159: note = nucleotide sequence encoding Streptococcus pyogenes Cas9 Protein with the N888R and A889Q mutations
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctatacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccatcgggtggacaagaagcctaatagaaagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgcggcagcatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaagaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggcccccccacatcattaagacaatcgcctccaagacccagagcattaagaagtacagcacagacattctgggcaacctgtatgaagtgaaatctaagaagcaccctcagatcatcaaaaagggc

SEQ ID NO: 50 - protein, 1053 aa, synthetic construct
  REGION 1..1053: note = Cas9 Protein with the N888G mutation
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLGAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 51 - DNA, 3159 bp, synthetic construct
  source 1..3159: mol_type = other DNA
  misc_feature 1..2661: note = nucleotide sequence encoding Streptococcus pyogenes Cas9 Protein with the N888G mutation
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctatacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccatcgggtggacaagaagcctaatagaaagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgggngcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaagaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggcccccccacatcattaagacaatcgcctccaagacccagagcattaagaagtacagcacagacattctgggcaacctgtatgaagtgaaatctaagaagcaccctcagatcatcaaaaagggc

(19) United States
(12) Patent Application Publication
(10) Pub. No.: US 2023/0295611 A1
(43) Pub. Date: Sep. 21, 2023

(54) CAS9 PROTEIN FOR GENOME EDITING

(71) Applicants: Versitech Limited, Hong Kong (CN); Centre of Oncology and Immunology Limited, Hong Kong (CN)

(72) Inventors: Siu Lun WONG, Hong Kong (CN); Dawn Gek Lian THEAN, Singapore (SG)

(21) Appl. No.: 18/176,819

(22) Filed: Mar. 1, 2023

Related U.S. Application Data
(60) Provisional application No. 63/268,745, filed on Mar. 1, 2022.

(51) Int. Cl. C12N 15/10; C12N 15/11; C12N 9/22

(52) U.S. Cl. CPC C12N 15/1082; C12N 15/1089; C12N 15/11; C12N 9/22; C12N 15/102; C12N 2310/20; C12N 2800/80

(57) ABSTRACT

The subject invention pertains to a Cas9 protein with an amino acid mutation at residues 888, 889, or a combination thereof of a WED domain and/or residues 988, 989, or a combination thereof of a PI domain. The subject invention can further pertain to a Cas9 protein with mutations at amino acid positions N986, D987, L988, L989, or any combination thereof. The subject invention also pertains to a method of enhancing the activity of KKH-SaCas9. In addition, a method of machine learning-based in silico screens for genome editing protein engineering is provided, including steps of populating a predictive machine learning model with an input dataset comprising empirical measurements of on-target activities of sgRNAs paired with a screening library of genome editing enzyme variants; running the predictive machine learning model with predefined parameters; and evaluating performance of the predictive machine learning model.

### CROSS-REFERENCE TO RELATED APPLICATION

This application claims the benefit of U.S. Provisional Application Ser. No. 63/268,745, filed Mar. 1, 2022, which is hereby incorporated by reference in its entirety including any tables, figures, or drawings.

### REFERENCE TO SEQUENCE LISTING

The Sequence Listing for this application is labeled “UHK273X.xml” which was created on May 9, 2023 and is 95,761 bytes. The entire content of the sequence listing is incorporated herein by reference in its entirety.

### BACKGROUND OF THE INVENTION

The CRISPR-associated protein 9 (Cas9) has become an important tool for genome editing. The CRISPR system includes several components where Cas9 specificity is guided by a single guide RNA (sgRNA) matching the complementary target DNA site, while the protospacer adjacent motif (PAM) lying proximal to the target DNA site is required for sequence-specific recognition. In particular, Staphylococcus pyrogenes Cas9 (SpCas9) is a well characterized enzyme that is popularly used for genome editing due to its short PAM 5′-NGG-3′ which is advantageous for broader genome editing applications and high average editing efficiency.

However, there are concerns regarding the higher off-target effects that may dampen the editing accuracy. Previous studies have been conducted to further modify SpCas9 to optimize editing accuracy and reduce constraints for PAM recognition¹⁻¹⁰. Nevertheless, it is very challenging to minimize the bulky nature of SpCas9, thereby limiting applications of SpCas9 for in vivo genome editing in which adeno-associated viruses having a packaging limit of about 4.5 kb are commonly used for clinical gene therapy.

Therefore, researchers have turned to the better characterized smaller Cas9 variants with activities comparable to SpCas9, such as the Staphylococcus aureus Cas9 (SaCas9)¹¹. Although SaCas9 is desirable for the packaging of genetic therapeutics, it also has certain drawbacks such as longer PAM 5′-NNGRRT-3′ and reduced genome coverage, leaving room for improvement for higher activity and specificity.

At present, most of the optimized Cas9 variants possess 2 to 7 mutations spanning multiple protein domains^{1-9,12-15} and each of the unique mutation combinations has contributed to comparable performance and editing fidelity. For example, >30 and >17 different amino-acid sites are engineered among the >13 SpCas9 and >8 SaCas9 variants, respectively. Nevertheless, the results represent only a small proportion of amino-acid sites interacting with the sgRNA-DNA complex^{16,17} each site being a potential candidate for optimization. However, a systematic experimental screen across all the candidate amino-acid positions to identify the best-performing Cas9 variants is both labor-intensive and prohibitively expensive. For instance, antibody maturation and viral capsid diversification involves a great number of fully saturated mutagenesis, ranging from 9 to 28 amino-acid sites. The capacity to evaluate such a large number of variants far exceeds what is experimentally feasible, even by massively parallel experiments.

Machine learning (ML) is advantageous in reducing the burden of experimental screen of protein engineering and in silico screens have shown great success in identifying high-performance variants of enzymes 18, optogenetic proteins¹⁹, binders²⁰, and viral capsids²¹. Previous studies have shown that the ML approach allows reliably prediction of the fitness of a full virtual library covering 10⁵-10¹² variants based on a small sub-sample of empirical fitness data of 10³-10⁴ variants or even less^{20,22}. Aiming to minimize the screening efforts, ML-guided approach such as machine learning-assisted approach to directed evolution (MLDE)^{23,24} extrapolates from the experimental determined fitness of a small sample of variants from a combinatorial mutant library to predict the full variant space covered by the multi-site saturation mutagenesis library in silico. Moreover, such approach is highly compatible with the existing screening platforms, which use fluorescence-activated cell sorting and next-generation sequencing as readouts, making it possible to evaluate the functionality of protein variants in a pooled library setting.

Although there were prior studies focusing on modifications of Pam-interacting (PI) domains in modifying around the PAM duplex region¹⁴, there is a lack of investigation on modification of the WED domain of SaCas9.

### BRIEF SUMMARY OF THE INVENTION

There continues to be a need in the art for improved Cas9 protein, and/or improved designs and techniques for methods and systems for a machine learning guided approach to meet the challenges of the optimization of Cas9.

In certain embodiments, the subject invention pertains to a Cas9 protein, according to SEQ ID NOs: 3 or 4 with an amino acid mutation at residues 888, 889, or a combination thereof of a WED domain and/or residues 988, 989, or a combination thereof of a PI domain. In certain embodiments, the mutation at residue 888 is N to Q, according to SEQ ID NO: 40; the mutation at residue 888 is N to Q and at residue 889 is A to S, according to SEQ ID NO: 41; the mutation at residue 888 is N to H and at residue 889 is A to Q, according to SEQ ID NO: 42; the mutation at residue 888 is N to S and at residue 889 is A to Q, according to SEQ ID NO: 43; the mutation at residue 888 is N to R and at residue 889 is A to Q, according to SEQ ID NO: 44; and/or the mutation at residue 888 is N to G, according to SEQ ID NO: 50. The subject invention can further pertain to a Cas9 protein with mutations at amino acid positions N986, D987, L988, L989, or any combination thereof.

Embodiments of the subject invention pertain to machine learning assisted methods and systems for engineering activity-enhanced Staphylococcus aureus Cas9's KKH variants for genome editing.

According to an embodiment of the subject invention, a method of machine learning-based in silico screens for genome editing is provided. The method comprises populating a predictive machine learning model with an input dataset comprising empirical measurements of on-target activities of sgRNAs, running the predictive machine learning model with predefined parameters, and evaluating performance of the predictive machine learning model. Moreover, enrichment scores of the empirical measurements are min-max normalized to scaled fitness scores ranging between 0 and 1. The input dataset includes empirical measurements of different percentages generated to test minimal number of inputs for effective selection of top variants by predictions of the machine learning model. Populating a predictive machine learning model with an input dataset further comprises generating a plurality of replicates of the input dataset based on a randomized selection scheme or a diverse selection scheme for variants. The generating a plurality of replicates based on the randomized selection scheme comprises randomly selecting a pre-defined number of enrichment scores. The generating a plurality of replicates based on the diverse selection scheme comprises keeping running randomly sampling variants with available enrichment scores until no variants sharing more than p 1-mismatch-neighbours and q 2-mismatches neighbors are present in the input dataset. Further, the predefined parameters comprise Belper and Georgiev embeddings of full-length amino-acid sequences of SpCas9 (UniProtKB—Q99ZW2 (CAS9_STRP1)) (SEQ ID NO: 3) and SaCas9 (UniProtKB—J7RUA5 (CAS9_STAAU)) (SEQ ID NO: 4) substituted with designated variant's amino-acid residue combination. The performance of the predictive machine learning model includes precision, specificity, and sensitivity of the embeddings of the predictive machine learning model. In addition, the evaluating performance of the predictive machine learning model comprises counting numbers of true positives, true negatives, false positives, and false negatives for each result and deriving metrics of the performance of the predictive machine learning model based on the numbers counted.

In another embodiment of the subject invention, a method combining machine learning-based in silico screens for genome editing with downstream structure-guided rational design is provided. The method comprises populating a predictive machine learning model with an input dataset comprising empirical measurements of on-target activities of sgRNAs, running the predictive machine learning model with predefined parameters, evaluating performance of the predictive machine learning model, constructing plasmid, cell culturing and transducing, conducting fluorescent protein disruption assays, performing immunoblot analysis, performing T7 endonuclease I assay, performing GUIDE-seq, and performing molecular dynamic simulations on the variants.

In certain embodiments of the subject invention, a computer program product is provided and comprises a non-transitory computer-executable storage device having computer readable program instructions embodied thereon that when executed by a computer cause the computer to perform machine learning-based in silico screens for genome editing. The computer-executable program instruction comprises populating a predictive machine learning model with an input dataset comprising empirical measurements of on-target activities of sgRNAs, running the predictive machine learning model with predefined parameters, and evaluating performance of the predictive machine learning model. Moreover, enrichment scores of the empirical measurements are min-max normalized to scaled fitness scores ranging between 0 and 1. The input dataset includes empirical measurements of different percentages generated to test minimal number of inputs for effective selection of top variants by predictions of the machine learning model. The populating a predictive machine learning model with an input dataset further comprises generating a plurality of replicates of the input dataset based on a randomized selection scheme or a diverse selection scheme for variants. The generating a plurality of replicates based on the randomized selection scheme comprises randomly selecting a pre-defined number of enrichment scores. The generating a plurality of replicates based on the diverse selection scheme comprises keeping running randomly sampling variants with available enrichment scores until no variants sharing more than p 1-mismatch-neighbours and q 2-mismatches neighbors are present in the input dataset. The predefined parameters comprise Belper and Georgiev embeddings of full-length amino-acid sequences of SpCas9 (UniProtKB—Q99ZW2 (CAS9_STRP1)) (SEQ ID NO: 3) and SaCas9 (UniProtKB—J7RUA5 (CAS9_STAAU)) (SEQ ID NO: 4) substituted with designated variant's amino-acid residue combination. The performance of the predictive machine learning model includes precision, specificity, and sensitivity of the embeddings of the predictive machine learning model. The evaluating performance of the predictive machine learning model comprises counting numbers of true positives, true negatives, false positives, and false negatives for each result and deriving metrics of the performance of the predictive machine learning model based on the numbers counted.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B show results of MLDE predicting the activity of SpCas9 at high precision, wherein FIG. 1A shows top variants identified when input of various sizes are supplied to MLDE based on Belper embedding and parameter 1 settings, variants with at least 70% wild-type activity identified in at least one of three replicates are highlighted in a color of tomato and shown with varying input sample sizes that represent 5% (33 variants), 10% (65 variants), 20% (130 variants), 50% (325 variants) and 70% (455 variants) of experimentally determined enrichment measurements, the heatmaps of 650 variants showing the empirical dataset that variants with at least 70% WT activity which are highlighted in the color of tomato, variants with missing on-target activity information are highlighted in grey and variants with lower than 70% wild-type activity are highlighted in black; and wherein FIG. 1B shows scatter plots demonstrating the enrichment, precision, sensitivity, and specificity of the MLDE (based on the Belper and parameter1 settings) on activity predictions with varying input data sizes represented by the x-axis with the three replicates of randomized variant selections, according to an embodiment of the subject invention.

FIG. 2 shows results of experimental screen and MLDE screen identifying activity-enhanced KKH-SaCas9 variants, the scatterplots showing on-target activities (E-scores) of variants based on the MLDE prediction (y-axis) and experimental screens over the full combinatorial mutant library of KKH-SaCas9 (x-axis), wherein three independent sgRNAs (sg1, sg2, sg3) are used, according to an embodiment of the subject invention.

FIGS. 3A-3H illustrate the improvement of structure-guided engineering in the editing efficiency of activity-enhanced KKH-SaCas9 variants, wherein FIG. 3A is a schematic representation of molecular modelling of N888Q and N888R/A889Q mutations on the WED domain of SaCas9 depicting their increased interactions with residues on its PI domain and the DNA backbone; FIGS. 3B-3E show results of KKH-SaCas9 variants carrying mutations on residues 888 and/or 889 that are individually constructed and characterized using GFP disruption assays with three independent sgRNAs, the editing efficiency of the KKH-SaCas9 variants being measured as the percentage of cells with depleted GFP fluorescence using flow cytometry; FIGS. 3F-3G show results of assessment of KKH-SaCas9 variants' on-target editing with sgRNAs targeting endogenous loci, the percentage of sites with indels being measured using a T7 endonuclease I (T7E1) assay, the ratios of the on-target activity of KKH-SaCas9 variants with N888Q and N888R/A889Q mutations to the activity of KKH-SaCas9 being determined, and the mean and standard deviation for the normalized percentage of indel formation being shown for the eight loci tested, each locus being measured twice; and FIG. 3H is a Western blot analysis illustrating similar protein expression between the KKH-SaCas9 variants, according to an embodiment of the subject invention.

FIGS. 4A-4E show that the activity-enhancing mutations increase activity of high-fidelity KKH-SaCas9-SAV2 variant while maintaining its high editing accuracy; wherein FIGS. 4A-4B show results of assessment of high-fidelity KKH-SaCas9 variants' on-target editing with sgRNAs targeting endogenous loci, the percentage of sites with indels being measured using a T7 endonuclease I (T7E1) assay, the ratio of the on-target activity of KKH-SaCas9-SAV2 with N888R/A889Q mutations to the activity of KKH-SaCas9-SAV2 being determined, and the mean and standard deviation for the normalized percentage of indel formation being shown for the eight loci tested, each locus being measured twice; FIG. 4C shows results of Western blot analysis on protein expression of the KKH-SaCas9-SAV2 variants; and FIGS. 4D-4E show GUIDE-seq genome-wide specificity profiles for KKH-SaCas9 and KKH-SaCas9-SAV2 variants with or without N888R/A889Q mutations, the number of off-target sites and the on-to-off target ratio being determined for each of the five independent sgRNAs used, the full dataset being presented in FIG. 10, according to an embodiment of the subject invention.

FIG. 5 shows results of performance of MLDE on SpCas9 activity predictions based on different embeddings, model parameters, and input datatypes, wherein boxplots demonstrate effects of the precision, specificity, and sensitivity of ML on SpCas9 activity using combinations of embedding (Belper/Georgiev) and model parameters, the MLDE predictions being evaluated from three replicates of 10%, 20%, 50% and 70% of input training data, the box summarizing 25, 50 and 75 quartiles, whiskers showing values within 1.5 times of interquartile ranges and dots being the outliners, according to an embodiment of the subject invention.

FIGS. 6A-6E show results of experimental screening of the activity of KKH-SaCas9 variants, wherein FIG. 6A is a schematic representation of the strategy for the profiling of the activities of KKH-SaCas9 variants in human cells, a library of 1,296 KKH-SaCas9 variants being assembled by PCR-based mutagenesis and being cloned in tandem with a gRNA targeting GFP expressed from a U6 promoter, the library being delivered via lentiviruses at a multiplicity of infection of about 0.3 to OVCAR8-ADR reporter cell lines in which the RFP and GFP genes are expressed from UBC and CMV promoters, respectively, fluorescent protein expressions being analyzed by flow cytometry and the results of analysis being shown in FIG. 6B, the activity of KKH-SaCas9 being measured by reporter systems in which the gRNA spacer sequence completely matches the GFP target site, cells with an active KKH-SaCas9 variant being expected to lose GFP fluorescence, cells being sorted into bins each encompassing about 5% of the population based on GFP fluorescence, and their genomic DNA being extracted for quantification of the variant by Illumina NovaSeq; and wherein FIGS. 6C-6E show scatterplots comparing the barcode count of each KKH-SaCas9 variant between bin A (GFP-negative) and bin B (GFP-positive) populations, each dot representing an KKH-SaCas9 variant, and wild-type (WT) KKH-SaCas9 being labelled, solid reference lines denoting two-fold enrichment, and the dotted reference line corresponding to no change in barcode count in the bin A as compared to the bin B population, three sgRNAs with permissive (sg1, sg2, sg3) being shown in FIG. 6C and three sgRNAs with non-permissive (sg5, sg6, sg7) being shown in FIG. 6E wherein PAMs for KKH-SaCas9 being used, bubble plot summarizing the enrichment scores determined for each KKH-SaCas9 variant with the three sgRNAs with permissive PAMs as shown in FIG. 6D, according to an embodiment of the subject invention.

FIG. 7 shows comparisons of MLDE prediction results and experimental screen data over KKH-SaCas9 variants with top 5% activities in the screening library, wherein the heatmaps show the occurrences of the amino-acid residues per site among the top 5% variants identified by the MLDE (left panels) and by experimental screens (right panels), and wherein three independent sgRNAs (sg1, sg2, sg3) are used, according to an embodiment of the subject invention.

FIG. 8 shows validation of the screen hits of activity-enhanced KKH-SaCas9 variants using non-pooled assays, wherein KKH-SaCas9 variants carrying mutations on residues 888 and/or 889 that are individually constructed and characterized using GFP disruption assays with three sgRNAs, the editing efficiency of the KKH-SaCas9 variants being measured as the percentage of cells with depleted GFP fluorescence using flow cytometry, according to an embodiment of the subject invention.

FIG. 9 shows schematic representations of molecular models of other variants being tested with mutations introduced to residues 888 and 889 at the WED domain of SaCas9, wherein the dotted lines denote the interactions modelled among the amino-acid residues of SaCas9, as well as those modelled among the amino-acid residues of SaCas9 and the target DNA's backbone, according to an embodiment of the subject invention.

FIG. 10 shows full datasets of GUIDE-seq genome-wide specificity profiles for KKH-SaCas9 and KKH-SaCas9-SAV2 variants with or without N888R/A889Q mutations, wherein mismatched positions in off-target sites are colored and GUIDE-seq read counts are used as a measurement of the cleavage efficiency at a given site, according to an embodiment of the subject invention.

FIG. 11 shows that the activity-enhancing mutations increase activity of high-fidelity KKH-SaCas9-SAV2 variant and generate reduced off-target edits at sites harboring sequences with single and double mismatch(es) to sgRNA spacer compared to wild-type, wherein cells expressing the KKH-SaCas9 variants are infected with lentiviruses encoding sgRNAs and carry no (i.e., GFPsg8) or one-base to two-base mismatch(es) against the target, the editing efficiency being measured as the percentage of cells with depleted GFP fluorescence using flow cytometry, and values reflecting the mean of two or three independent biological replicates, according to an embodiment of the subject invention.

FIGS. 12A and 12B show schematic representations of strategies of using MLDE to expand the number of mutation sites surveyed, wherein FIG. 12A shows multiple smaller focused libraries with mutagenesis up to 6 sites (highlighted in light blue) with 1-2 sites in common to another library being constructed, the empirical data of all 7 screens being combined and fed into MLDE to identify the best variants across all of the sites; and wherein FIG. 12B shows performing iterative rounds of targeted mutagenesis and MLDE, up to 6 sites (highlighted in light blue), each with a few candidate residues being selected from structure-guided design, being screened in a library, the top-performing variants predicted by MLDE from each round seeding the mutagenesis library of the next round with a new set of amino-acid sites subjected to mutagenesis, until a high performance variant is identified, according to an embodiment of the subject invention.

FIGS. 13A-13D show results of evaluation of performance of MLDE on SpCas9-sg8ON activity, wherein FIG. 13A shows top variants identified when input of various sizes are supplied to MLDE based on Belper embedding+parameter 1 settings, variants (highlighted in a color of tomato) with at least 70% wild-type activity identified in at least 1 of the 3 replicates are shown for sg8ON with various input sample sizes that represent 10% (73), 20% (146), 50% (365) and 70% (510) of experimentally determined enrichment measures, the heatmaps at the last column (sg8ON— 729 variants) showing the empirical dataset that variants with at least 70% WT activity are highlighted in a color of tomato, variants with missing on-target activity information being highlighted in grey and variants with lower than 70% wild-type activity being highlighted in black; wherein FIG. 13B shows boxplots reporting the precision, specificity, and sensitivity of ML on SpCas9-sg8ON activity based on combinations of embedding (Belper/Georgiev) and model parameters, the MLDE predictions being evaluated from three replicates of 10%, 20%, 50% and 70% of input training data, the box summarizing the 25, 50 and 75 quartiles, whiskers showing values within 1.5 times of interquartile ranges and dots being the outliners; wherein FIG. 13C shows histograms of the distribution of normalized fitness values of the SpCas9-sg50N and sg8ON activity datasets, dash-line indicating the 70% wild-type activity threshold used for labelling variants as positives and negatives; and wherein FIG. 13D shows results of evaluation of performance of MLDE on SpCas9-sg8ON activity after setting floor activity as −3, the MLDE predictions being evaluated from three replicates of 10%, 20%, 50% and 70% of randomly selected input training data, extreme values being removed by setting enrichment score no lower than −3, the boxplots reporting the precision, sensitivity and specificity of ML on sg8ON activities based on combinations of embedding (Belper/Georgiev) and model parameters with or without removing the extremely low E-scores, the box summarizing the 25, 50 and 75 quartiles, whiskers showing values within 1.5 times of interquatile ranges and dots being the outliners, according to an embodiment of the subject invention.

FIG. 14 illustrates the validation of another activity enhancing mutation using the GFP disruption reporter system with three GFP sgRNAs; sgRNA1, sgRNA 2 and sgRNA4. This new variant N888G (or “GAL”) was identified through conducting saturation mutagenesis on amino acid positions 888 and 889.

### BRIEF DESCRIPTION OF THE SEQUENCES

SEQ ID NO: 1: dsODN oligonucleotide

SEQ ID NO: 2: dsODN oligonucleotide

SEQ ID NO: 3: SaCas9 (UniProtKB—Q99ZW2 (CAS9_STRP1)) amino acid sequence

SEQ ID NO: 4: SaCas9 (UniProtKB—J7RUA5 (CAS9_STAAU)) amino acid sequence

SEQ ID NO: 5: GFPsg1 protospacer

SEQ ID NO: 6: GFPsg2 protospacer

SEQ ID NO: 7: GFPsg3 protospacer

SEQ ID NO: 8: GFPsg4 protospacer

SEQ ID NO: 9: GFPsg5 protospacer

SEQ ID NO: 10: GFPsg6 protospacer

SEQ ID NO: 11: GFPsg7 protospacer

SEQ ID NO: 12: GFPsg8 protospacer

SEQ ID NO: 13: EMX1_sg1 protospacer

SEQ ID NO: 14: EMX1_sg4 protospacer

SEQ ID NO: 15: EMX1_sg6 protospacer

SEQ ID NO: 16: EMX1_sg10 protospacer

SEQ ID NO: 17: EMX1_sg2 protospacer

SEQ ID NO: 18: EMX1_sg7 protospacer

SEQ ID NO: 19: VEGFA_sg8 protospacer

SEQ ID NO: 20: AAVS1_sg4 protospacer

SEQ ID NO: 21: CCR5_sg2 protospacer

SEQ ID NO: 22: EMX1_sg1 forward primer

SEQ ID NO: 23: EMX1_sg1 reverse primer

SEQ ID NO: 24: EMX1_sg4 forward primer

SEQ ID NO: 25: EMX1_sg4 reverse primer

SEQ ID NO: 26: EMX1_sg6 forward primer

SEQ ID NO: 27: EMX1_sg6 reverse primer

SEQ ID NO: 28: EMX1_sg10 forward primer

SEQ ID NO: 29: EMX1_sg10 reverse primer

SEQ ID NO: 30: EMX1_sg2 forward primer

SEQ ID NO: 31: EMX1_sg2 reverse primer

SEQ ID NO: 32: EMX1_sg7 forward primer

SEQ ID NO: 33: EMX1_sg7 reverse primer

SEQ ID NO: 34: VEGFA_sg8 forward primer

SEQ ID NO: 35: VEGFA_sg8 reverse primer

SEQ ID NO: 36: AAVS1_sg4 forward primer

SEQ ID NO: 37: AAVS1_sg4 reverse primer

SEQ ID NO: 38: CCR5_sg2 forward primer

SEQ ID NO: 39: CCR5_sg2 reverse primer

SEQ ID NO: 40: Cas9 Protein with the N888Q mutation

SEQ ID NO: 41: Cas9 Protein with the N888Q and A889S mutations

SEQ ID NO: 42: Cas9 Protein with the N888H and A889Q mutations

SEQ ID NO: 43: Cas9 Protein with the N888S and A889Q mutations

SEQ ID NO: 44: Cas9 Protein with the N888R and A889Q mutations

SEQ ID NO: 45: Nucleotide sequence encoding Cas9 Protein with the N888Q mutation

SEQ ID NO: 46: Nucleotide sequence encoding Cas9 Protein with the N888Q and A889S mutations

SEQ ID NO: 47: Nucleotide sequence encoding Cas9 Protein with the N888H and A889Q mutations

SEQ ID NO: 48: Nucleotide sequence encoding Cas9 Protein with the N888S and A889Q mutations

SEQ ID NO: 49: Nucleotide sequence encoding Cas9 Protein with the N888R and A889Q mutations

SEQ ID NO: 50: Cas9 Protein with the N888G mutations

SEQ ID NO: 51: Nucleotide sequence encoding Cas9 Protein with the N888G mutation

### DETAILED DISCLOSURE OF THE INVENTION

Embodiments of the subject invention are directed to machine learning assisted methods and systems for engineering activity-enhanced Staphylococcus aureus Cas9's KKH variants for genome editing.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not prelude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e. the value can be +/−10% of the stated value. For example, “about 1 kg” means from 0.90 kg to 1.1 kg.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

In this application, the terms “polypeptide”, “peptide”, and “protein” are used interchangeably herein to refer to a polymer of amino acids. The terms apply to amino acid polymers in which one or more amino acid residues are artificial chemical mimetic of a corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used in herein, the terms “identical” or percent “identity”, in the context of describing two or more polynucleotide or amino acid sequences, refer to two or more sequences or subsequences that are the same or have a specified percentage of amino acid residues or nucleotides that are the same (for example, a variant protein used in the method of this invention has at least 80% sequence identity, preferably 85%, 90%, 91%, 92%, 93, 94%, 95%, 96%, 97%, 98%, 99%, or 100% identity, to a reference sequence), when compared and aligned for maximum correspondence over a comparison window, or designated region as measured using one of the following sequence comparison algorithms or by manual alignment and visual inspection. Such sequences are then said to be “substantially identical”. With regard to polynucleotide sequences, this definition also refers to the complement of a test sequence. The comparison window, in certain embodiments, refers to the full-length sequence of a given polypeptide, for example a specific enzyme.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Machine learning (ML) can be applied to a focused library derived from the structure-guided design. Such focused library generally targets multiple sites, for example, eight sites for SpCas9 optimization, that are keys to the protein functionality with deliberated mutations that are restricted to a few residues per site. It is demonstrated that the ML-based in silico screens are efficient and accurate in independent Cas9 optimization tasks, resulting in a reduction of the wet-lab labor by as much as 90%. Further, activities of SaCas9 are boosted whilst broader PAM specificities are obtained. The modifications based on the E782K/N968K/R1015H SaCas9 variant (KKH-SaCas9) lead to activities comparable with wild-type SaCas9 and recognition of an expanded PAM 5′-NNNRRT-3′¹³.

By combining ML-based and combinatorial mutagenesis screens with downstream structure-guided rational design and wet-lab validations, changes in the WED domain can provide stronger interactions with the PI domain, thereby increasing the DNA-binding ability of KKH-SaCas9 protein. The results reveal that the modification of the WED domain may come through more often in enhancing the protein's activity rather than the changes in the PI domain. In addition, the same set of mutations can be tested with a high-fidelity SaCas9 variant, KKH-SaCas9-SAV2, indicating that the mutations may have wide applications. The work flow and associated parameters of the ML approach can be configured to maximize its effectiveness in succeeding screens for engineering other components of the Cas9 system and for gene editing.

In one embodiment, a method of machine learning-based in silico screens for genome editing is provided. The method comprises populating a predictive machine learning model with an input dataset comprising empirical measurements of on-target activities of sgRNAs; running the predictive machine learning model with predefined parameters; and evaluating performance of the predictive machine learning model. The enrichment scores of the empirical measurements are min-max normalized to scaled fitness scores ranging between 0 and 1. The input dataset includes empirical measurements of different percentages generated to test minimal number of inputs for effective selection of top variants by predictions of the machine learning model. The populating a predictive machine learning model with an input dataset further comprising generating a plurality of replicates of the input dataset based on a randomized selection scheme or a diverse selection scheme for variants. The generating a plurality of replicates based on the randomized selection scheme comprises randomly selecting a pre-defined number of enrichment scores. The generating a plurality of replicates based on the diverse selection scheme comprising keeping running randomly sampling variants with available enrichment scores until no variants sharing more than p 1-mismatch-neighbours and q 2-mismatches neighbors are present in the input dataset. The predefined parameters comprise Belper and Georgiev embeddings of full-length amino-acid sequences of SpCas9 (UniProtKB—Q99ZW2 (CAS9_STRP1)) (SEQ ID NO: 3) and SaCas9 (UniProtKB—J7RUA5 (CAS9_STAAU)) (SEQ ID NO: 4) substituted with designated variant's amino-acid residue combination. The performance of the predictive machine learning model includes precision, specificity, and sensitivity of the embeddings of the predictive machine learning model. The evaluating performance of the predictive machine learning model comprises counting numbers of true positives, true negatives, false positives, and false negatives for each result and deriving metrics of the performance of the predictive machine learning model based on the numbers counted.

In another embodiment, a method combining machine learning-based in silico screens for genome editing with downstream structure-guided rational design is provided. The method comprises populating a predictive machine learning model with an input dataset comprising empirical measurements of on-target activities of sgRNAs; running the predictive machine learning model with predefined parameters; evaluating performance of the predictive machine learning model; constructing plasmid; cell culturing and transducing; conducting fluorescent protein disruption assays; performing immunoblot analysis; performing T7 endonuclease I assay; performing GUIDE-seq; and performing molecular dynamic simulations on the variants.

In another embodiment, a computer program product comprising a non-transitory computer-executable storage device having computer readable program instructions embodied thereon that when executed by a computer cause the computer to perform machine learning-based in silico screens for genome editing is provided. The computer-executable program instruction comprises populating a predictive machine learning model with an input dataset comprising empirical measurements of on-target activities of sgRNAs; running the predictive machine learning model with predefined parameters; and evaluating performance of the predictive machine learning model. The enrichment scores of the empirical measurements are min-max normalized to scaled fitness scores ranging between 0 and 1. The input dataset includes empirical measurements of different percentages generated to test minimal number of inputs for effective selection of top variants by predictions of the machine learning model. The populating a predictive machine learning model with an input dataset further comprising generating a plurality of replicates of the input dataset based on a randomized selection scheme or a diverse selection scheme for variants. The generating a plurality of replicates based on the randomized selection scheme comprises randomly selecting a pre-defined number of enrichment scores. The generating a plurality of replicates based on the diverse selection scheme comprising keeping running randomly sampling variants with available enrichment scores until no variants sharing more than p 1-mismatch-neighbours and q 2-mismatches neighbors are present in the input dataset. The predefined parameters comprise Belper and Georgiev embeddings of full-length amino-acid sequences of SpCas9 (UniProtKB—Q99ZW2 (CAS9_STRP1)) (SEQ ID NO: 3) and SaCas9 (UniProtKB—J7RUA5 (CAS9_STAAU)) (SEQ ID NO: 4) substituted with designated variant's amino-acid residue combination. The performance of the predictive machine learning model includes precision, specificity, and sensitivity of the embeddings of the predictive machine learning model. The evaluating performance of the predictive machine learning model comprises counting numbers of true positives, true negatives, false positives, and false negatives for each result and deriving metrics of the performance of the predictive machine learning model based on the numbers counted. The plasmid is obtained by polymerase chain reaction (PCR), restriction enzyme digestion, ligation, one-pot ligation, Gibson assembly, or a combination thereof.

### Methods

### 1. Generation of Data Input for the MLDE Model

The previously published SpCas9 data⁸ surveying the on-target activity of sg50N (650 empirical data points) that target a red fluorescent protein (RFP) sequence as the input data are used for the MLDE model. The enrichment scores (E-scores) are min-max normalized to the scaled fitness scores ranging between 0 and 1.

In one embodiment, input datasets including 10%, 20%, 50%, and 70% of empirical measurements are generated to test the minimal number of input for effective selection of top variants from the MLDE prediction, corresponding to datasets of 65, 130, 325, and 445 empirically measured on-target activities. Three replicates are generated for each size, subjected to either randomized or diverse selection schemes for variants. To generate the randomized dataset, the sample_n( ) function from dplyr in R to randomly select the pre-defined number of E-scores is utilized. In order to generate the diverse dataset, randomly sampling variants with available E-scores are kept running until no variants sharing more than p 1-mismatch-neighbors and q 2-mismatches neighbors are present in the input dataset. The thresholds p and q for each dataset can be found in Table 1 below.

TABLE 1
Threshold p and q for each dataset

| | | Percentage of | Number of 1- | Number of 2- |
|---|---|---|---|---|
| | | empirical | mismatch- | mismatch- |
| | Input | measurements | neighbours | neighbours |
| sgRNA | datapoint | (%) | (p) | (q) |
| sg5 | 65 | 10 | 1 | 1 |
| | 130 | 20 | 3 | 5 |
| | 325 | 50 | 5 | 14 |
| | 445 | 70 | 6 | 20 |
| sg8 | 73 | 10 | 1 | 2 |
| | 146 | 20 | 2 | 6 |
| | 365 | 50 | 6 | 17 |
| | 510 | 70 | 7 | 22 |

The MLDE model is run according to the default parameters. The Belper and Georgiev embedding of the full-length amino-acid sequences of SpCas9 (UniProtKB—Q99ZW2 (CAS9_STRP1)) and SaCas9 (UniProtKB—J7RUA5 (CAS9_STAAU)) substituted with the designated variant's amino-acid residue combination are applied. The MLDE GenerateEncodings.py is modified such that it processes a customized input fasta file containing the protein sequences of all the variants designed in the SpCas9 as well as the SaCas9 dataset rather than generating the full set of saturated mutagenesis variants. The MLDE ExecuteMlde.py is run with default parameters on the Belper and the Georgiev embeddings and with two different sets of parameters. Other default parameters include 5-fold cross validation and the top 3 models are used to average to get final prediction results. They are assigned as parameters 1 and 2, parameter 1 using the neural network models such as “OneHidden”, “TwoHidden”, “OneConv” and “TwoConv” available in the MLDE models, each with 20 rounds of hyperparameter optimization, while parameter 2 using fewer complex models such as “Linear-Tweedie”, “RandomForestRegressor”, “LinearSVR” and “ElasticNet”, each with 50 rounds of hyperparameter optimization.

The performance of parameters of the ML algorithm including precision, specificity, and sensitivity of the embeddings of the ML is then evaluated. In particular, variants with at least 70% of the wild-type activity are assigned as positives and the rest as negatives. Thus, true positives are variants with at least 70% activity of the wild-type, when being empirically tested with the sgRNA. Otherwise, they are determined to be true negatives. For each MLDE result, the positives and negatives are also labelled using the 70% wild-type activity threshold. Then, the numbers of true positives (TP), true negatives (TN), false positives (FP), and false negatives (FN) are counted for each result and the performance metrics are derived according to the formulas below:

$$specificity = \frac{T N}{T N + F P}$$

$$sensitivity = \frac{T P}{T P + F N}$$

$$Precision = \frac{T P}{T P + F P}$$

Another performance metric, enrichment, proposed by Sarfati et al.⁴² is also applied. The enrichment as determined by the equation below reveals the ratio of identifying true top 5% of hits when using the ML prediction for the random selection (“the null background”),

$$Enrichment = {I}_{S}^{prediction} / {I}_{S}^{random} = \frac{400 * {I}_{S}^{prediction}}{N}$$

where N is the total size of the test set and is the number of all the variants in the prediction in this case.

The input data handling, statistical analyses and graph plotting are carried out by R programs using packages ggplot2, tidyverse, readxl, Cairo, and stringdist.

2. Plasmid Construction The plasmids generated from the test results as shown in Table 2 below are obtained by standard molecular cloning techniques such as polymerase chain reaction (PCR), restriction enzyme digestion, ligation, one-pot ligation, or Gibson assembly. Customized oligonucleotides are ordered through Genewiz. Vectors are transformed into E. coli strain DH5α competent cells and selected with ampicillin (for example, 100 mg/ml, USB) or carbenicillin (for example, 50 mg/ml, Teknova). DNAs are extracted and purified by Plasmid Mini (for example, from Takara and Tiangen) or Midi preparation (for example, from QIAGEN) kits and sequences of the vectors are verified by Sanger sequencing.

TABLE 2
This file contains a list of constructs used in this work

| Construct ID | Design | Reference |
|---|---|---|
| pAWp9 | pFUGW-UBCp-RFP-CMVp-GFP | Wong et al., |
| | | PNAS, 2016; |
| | | 113(9): 2544-9 |
| AWp112 | pBT264-BsaI-BglII-U6-BbsIx2-sgRNA | This study |
| | scaffold-EcoRI-BsaI | |
| AWp124 | pFUGW-EFS-humanSaCas9(E782K, N968K, R1015H)- | This study |
| | NLS-T2A-modBFP | |
| DTp2 | pFUGW-EFS-humanSaCas9(E782K-Esp3Ix2-R1015H)- | This study |
| | NLS-T2A-modBFP | |
| DTp4a | pFUGW-EFS-humanSaCas9(E782K-Esp3Ix2-R1015H)- | This study |
| | NLS-T2A-modBFP-U6-GFPsg1-sgRNA scaffold | |
| DTp4b | pFUGW-EFS-humanSaCas9(E782K-Esp3Ix2-R1015H)- | This study |
| | NLS-T2A-modBFP-U6-GFPsg4-sgRNA scaffold | |
| DTp4c | pFUGW-EFS-humanSaCas9(E782K-Esp3Ix2-R1015H)- | This study |
| | NLS-T2A-modBFP-U6-GFPsg3-sgRNA scaffold | |
| DTp4d | pFUGW-EFS-humanSaCas9(E782K-Esp3Ix2-R1015H)- | This study |
| | NLS-T2A-modBFP-U6-GFPsg2-sgRNA scaffold | |
| DTp4g | pFUGW-EFS-humanSaCas9(E782K-Esp3Ix2-R1015H)- | This study |
| | NLS-T2A-modBFP-U6-GFPsg5-sgRNA scaffold | |
| DTp4i | pFUGW-EFS-humanSaCas9(E782K-Esp3Ix2-R1015H)- | This study |
| | NLS-T2A-modBFP-U6-GFPsg6-sgRNA scaffold | |
| DTp4j | pFUGW-EFS-humanSaCas9(E782K-Esp3Ix2-R1015H)- | This study |
| | NLS-T2A-modBFP-U6-GFPsg7-sgRNA scaffold | |
| DTp47A | pFUGW-EFS-humanSaCas9(Y239H, N419D, R654A, | This study |
| (SAV2 + | G655A, E782K, N888R, A889Q, N968K, R1015H)- | |
| R888Q889) | NLS-T2A-modBFP | |
| DTp52 | pFUGW-EFS-humanSaCas9(Y239H, N419D, R654A, | This study |
| (SAV2) | G655A, E782K, N968K, R1015H)-NLS-T2A-modBFP | |
| ZRp7b | pFUGW-UBCp-RFP-CMVp-GFP-U6p-GFPsg8-M1 | This study |
| pPZp112-M1 | pFUGW-UBCp-RFP-CMVp-GFP-U6p-GFPsg1-M1 | This study |
| pPZp112-M2 | pFUGW-UBCp-RFP-CMVp-GFP-U6p-GFPsg1-M2 | This study |
| pPZp112-M3 | pFUGW-UBCp-RFP-CMVp-GFP-U6p-GFPsg1-M3 | This study |
| pPZp112-M4 | pFUGW-UBCp-RFP-CMVp-GFP-U6p-GFPsg1-M4 | This study |
| pPZp112-M5 | pFUGW-UBCp-RFP-CMVp-GFP-U6p-GFPsg1-M5 | This study |
| pPZp112-M6 | pFUGW-UBCp-RFP-CMVp-GFP-U6p-GFPsg1-M6 | This study |
| pPZp112-M7 | pFUGW-UBCp-RFP-CMVp-GFP-U6p-GFPsg1-M7 | This study |
| pPZp112-M8 | pFUGW-UBCp-RFP-CMVp-GFP-U6p-GFPsg1-M8 | This study |
| pPZp112-M9 | pFUGW-UBCp-RFP-CMVp-GFP-U6p-GFPsg1-M9 | This study |
| pPZp112-M10 | pFUGW-UBCp-RFP-CMVp-GFP-U6p-GFPsg1-M10 | This study |
| pPZp112-M11 | pFUGW-UBCp-RFP-CMVp-GFP-U6p-GFPsg1-M11 | This study |
| pPZp112-M12 | pFUGW-UBCp-RFP-CMVp-GFP-U6p-GFPsg1-M12 | This study |
| pPZp112-M13 | pFUGW-UBCp-RFP-CMVp-GFP-U6p-GFPsg1-M13 | This study |
| pPZp112-M14 | pFUGW-UBCp-RFP-CMVp-GFP-U6p-GFPsg1-M14 | This study |
| pPZp112-M15 | pFUGW-UBCp-RFP-CMVp-GFP-U6p-GFPsg1-M15 | This study |
| pPZp112-M16 | pFUGW-UBCp-RFP-CMVp-GFP-U6p-GFPsg1-M16 | This study |
| pPZp112-M17 | pFUGW-UBCp-RFP-CMVp-GFP-U6p-GFPsg1-M17 | This study |
| pPZp112-M18 | pFUGW-UBCp-RFP-CMVp-GFP-U6p-GFPsg1-M18 | This study |
| pPZp112-M19 | pFUGW-UBCp-RFP-CMVp-GFP-U6p-GFPsg1-M19 | This study |
| pPZp112-M20 | pFUGW-UBCp-RFP-CMVp-GFP-U6p-GFPsg1-M20 | This study |

Next, storage vectors AWp28 (for example, Addgene #73850) and AWp112 are used to assemble the sgRNA chosen to target a specific gene and the sgRNA sequences employed are listed in Table 3 below. Oligonucleotide pairs of the sgRNA target sequences with BbsI sticky ends are then synthesized, annealed, and cloned into the BbsI-digested storage vector using T4 DNA ligase (for example, from New England Biolabs).

TABLE 3
This file contains a list of gRNA
protospacer sequences used in this study

| sgRNA ID | sgRNA protospacer sequence (*) |
|---|---|
| GFPsg1 | GGGACGGCGACGTAAACGGCC (SEQ ID NO: 5) |
| GFPsg2 | GGGCGAGGAGCTGTTCACCGG (SEQ ID NO: 6) |
| GFPsg3 | GCAACATCCTGGGGCACAAGC (SEQ ID NO: 7) |
| GFPsg4 | GGCGTGTCCGGCGAGGGCGAG (SEQ ID NO: 8) |
| GFPsg5 | GCTCGGCGCGGGTCTTGTAGT (SEQ ID NO: 9) |
| GFPsg6 | GGAACTTCACCTCGGCGCGGG (SEQ ID NO: 10) |
| GFPsg7 | GCACGGGGCCGTCGCCGATGG (SEQ ID NO: 11) |
| GFPsg8 | CACCTACGGCAAGCTGACCC (SEQ ID NO: 12) |
| EMX1_sg1 | GTGTGGTTCCAGAACCGGAGGA (SEQ ID NO: 13) |
| EMX1_sg4 | GCTCAGCCTGAGTGTTGAGGC (SEQ ID NO: 14) |
| EMX1_sg6 | GCAACCACAAACCCACGAGGG (SEQ ID NO: 15) |
| EMX1_sg10 | GGCTCTCCGAGGAGAAGGCCA (SEQ ID NO: 16) |
| EMX1_sg2 | TGGCCAGGCTTTGGGGAGGCC (SEQ ID NO: 17) |
| EMX1_sg7 | GGCCAGGCTTTGGGGAGGCC (SEQ ID NO: 18) |
| VEGFA_sg8 | GGGTGAGTGAGTGTGTGCGTG (SEQ ID NO: 19) |
| AAVS1_sg4 | GACTAGGAAGGAGGAGGCCT (SEQ ID NO: 20) |
| CCR5_sg2 | GTTGCCCTAAGGATTAAATGA (SEQ ID NO: 21) |

To prepare the lentiviral vector for SaCas9 variant expression, the AWp124 vector is modified via Gibson assembly to remove all existing Esp3J enzyme sites. Esp3J sites are then re-introduced flanking the PI and WED regions to incorporate the intended mutations, giving the DTp2 vector. To insert the sgRNA expression cassette, they are amplified from the storage vector by flanking BamHI and EcoRI (for example, from Thermo Fisher Scientific) sites to and ligated with the digested lentiviral vector DTp2. To generate the PI and WED mutations, oligonucleotides with the WED domain mutations are pooled with a 1:1 ratio as the forward primer, and the same procedure is applied to the PI domain for the reverse primer. PCR amplifications are carried out by the pooled forward and reverse primers with the original KKH-SaCas9 template to create the pooled mutations. By a one-pot ligation method, the pooled mutations are inserted into the Esp3I sites of DTp2. Moreover, the EFS promoter, together with a fluorescent protein expression from the downstream T2A-BFP, drives the SaCas9 expression. To create SaCas9-KKH-SAV2-plus (DTp47A), the Esp3I sites similarly done with DTp2 are incorporated into SaCas9-KKH-SAV2 (DTp52) via Gibson assembly, and then with one-pot ligation inserted the ‘plus’ mutations that are the N888R/A889Q. In conducting saturation mutagenesis on positions 888 and 889, amplifications were done using oligonucleotides designed with ‘NNS’ nucleotides for both positions and incorporated into the lentivectors with the appropriate gRNAs using the similar technique as described above.

### 3. Cell Culture and Transduction

HEK293T cells obtained from American Type Culture Collection (ATCC) and MHCC97L-Luc cells are maintained in Dulbecco's Modified Eagle Medium (DMEM) supplemented with 1×antibiotic-antimycotic and 10% FBS (for example, from Thermo Fisher Scientific). OVCAR8-ADR cells are maintained in RPMI 1640 medium supplemented with 10% FBS (for example, from Gibco). The HEK293T cells are used for lentiviral production for KKH-SaCas9 variant expression and for generating stable cell lines. The OVCAR8-ADR cells are transduced with a pAWp9 vector (for example, Addgene #73851) expressing RFP and GFP gene, driven by the hUbCp and CMV promoters, respectively, for the initial screening of KKH-SaCas9 pooled variants and for further validation. OVCAR8-ADR cells are also transduced with lentiviruses encoding RFP and GFP genes expressed from UBC and CMV promoters, respectively, and a tandem U6 promoter-driven expression cassette of sgRNA targeting the GFP site. For the initial screening, the KKH-SaCas9 variants are expressed with sgRNA targeting GFP using EFS and U6 promoters, respectively, followed by a T2A-BFP to determine KKH-SaCas9 expression. The cells are sorted with a Becton Dickinson BD Influx cell sorter. With the mutational screening, the KKH-SaCas9 selected variants are transduced into the stable OVCAR8-ADR cell lines harboring the GFP, RFP genes, and sgRNA. The MHCC97L-Luc cell lines are transduced to create the stable expression of the selected KKH-SaCas9 variants for the T7E1 and Guide-seq experiments. The cells are regularly tested and show negative for mycoplasma contamination. Lentivirus production and transduction are carried out as previously described⁸.

### 4. Fluorescent Protein Disruption Assay

Fluorescent protein disruption assays are conducted to determine DNA cleavage and indel-mediated disruption at the target site of the fluorescent protein, GFP, by the KKH-SaCas9 variants with the gRNA expressions, resulting in loss of cell fluorescence. The stable cell lines integrated with the GFP and RFP reporter gene, expressing the SaCas9 variants and sgRNA are washed, then resuspended with 1×PBS supplemented with 2% heat-inactivated FBS, and analyzed with Becton Dickinson LSR Fortessa Analyzer or ACEA NovoCyte Quanteon. Cells are gated on forward and side scatter, and at least 1×10⁴ cells are recorded per sample for each data set.

### 5. Immunoblot Analysis

Immunoblots are carried out as previously described⁸. Anti-SaCas9 (for example, 1:1,000, Cell Signaling #85687) and anti-GAPDH (for example, 1:5,000, Cell Signaling #2118) primary antibodies are used, followed by HRP-linked anti-mouse IgG (for example, 1:10,000, Cell Signaling #7076) and HRP-linked anti-rabbit IgG (for example, 1:20,000, Cell Signaling #7074) secondary antibodies.

### 6. T7 Endonuclease I Assay

T7 endonuclease I assay is performed as previously described to quantify the Cas9-induced mutagenesis in endogenous loci⁸. The targeted loci are amplified from 15-30 ng of genomic DNA extracted using DNeasy Blood and Tissue Kit (for example, from QIAGEN) using the primers as listed in Table 4 below. Quantification is based on relative band intensities measured using ImageJ. The editing efficiency is estimated by the formula, 100×(1−(1−(b+c)/(a+b+c))=) as previously described 43, where a is the integrated intensity of the uncleaved PCR product, and b and c are the integrated intensities of each cleavage product, respectively.

TABLE 4
This table contains a list of primers and
PCR conditions used for T7E1 assay

| Target | Forward primer | Reverse primer |
|---|---|---|
| gene | (5′ to 3′) | (5′ to 3′) |
| EMX1_sg1 | GGAGCAGCTGGTCAGAGG | CCATAGGGAAGGGGGACACTG |
| | GG (SEQ ID NO: 22) | G (SEQ ID NO: 23) |
| EMX1_sg4 | GGAGCAGCTGGTCAGAGG | CCATAGGGAAGGGGGACACTG |
| | GG (SEQ ID NO: 24) | G (SEQ ID NO: 25) |
| EMX1_sg6 | GGAGCAGCTGGTCAGAGG | CCATAGGGAAGGGGGACACTG |
| | GG (SEQ ID NO: 26) | G (SEQ ID NO: 27) |
| EMX1_sg10 | GGAGCAGCTGGTCAGAGG | CCATAGGGAAGGGGGACACTG |
| | GG (SEQ ID NO: 28) | G (SEQ ID NO: 29) |
| EMX1_sg2 | GGAGCAGCTGGTCAGAGG | CCATAGGGAAGGGGGACACTG |
| | GG (SEQ ID NO: 30) | G (SEQ ID NO: 31) |
| EMX1_sg7 | GGAGCAGCTGGTCAGAGG | CCATAGGGAAGGGGGACACTG |
| | GG (SEQ ID NO: 32) | G (SEQ ID NO: 33) |
| VEGFA_sg8 | TCCAGATGGCACATTGTC | AGGGAGCAGGAAAGTGAGGT |
| | AG (SEQ ID NO: 34) | (SEQ ID NO: 35) |
| AAVS1_sg4 | ACACCTAGGACGCACCAT | CTTGCTTTCTTTGCCTGGAC |
| | TC (SEQ ID NO: 36) | (SEQ ID NO: 37) |
| CCR5_sg2 | CCGGCCATTTCACTCTGA | TTGCTGCTAGCTTCCCTGTC |
| | CT (SEQ ID NO: 38) | (SEQ ID NO: 39) |

### 7. GUIDE-seq

GUIDE-seq is performed as previously described⁸. Approximately 1.6 million MHCC97L cells stably expressing the KKH-SaCas9 variants are transduced with sgRNAs. After 72 hours, electroporation is conducted according to the manufacturer's protocol using 1,100 pmol freshly annealed end-protected dsODN with 100 μl Neon tips (for example, from ThermoFisher Scientific). The dsODN oligonucleotides used are 5′-P-G*T*TTAATTGAGTTGTCATATGTTAATAACGGT*A*T-3′ (SEQ ID NO: 1) and 5′-P-A*T*ACCGTTATTAACATATGACAACTCAATTAA*A*C-3′ (SEQ TD NO: 2), where P represents 5′ phosphorylation and the asterisks indicate phosphorothioate linkages. Electroporation voltage, width and number of pulses are set to be 1100 V, 20 ins, and 3 pulses, respectively. Cells are harvested at day 7 post transduction of the sgRNA. Genomic DNA is extracted using DNeasy Blood and Tissue Kit (for example, from QIAGEN) according to the manufacturer's protocol. The gDNA collected for the SaCas9 variant and the sgRNA are sequenced on Illumina NextSeq System and analyzed by GUIDE-seq software⁴⁴.

### 8. Molecular Modelling

Molecular dynamic simulations are conducted on the variants using DynaMut³⁷. The variants mutations are singly inputted into the webserver and the structural outputs are then aligned with the crystal structure of SaCas9 (PDB: 5CZZ) on PyMol. The predicted rotamer of the mutations as indicated by DynaMut is subsequently used to replace the amino acid positions on the SaCas9 crystal structure. The predicted interactions determined by DynaMut and Pymol are indicated on the crystal structure to provide a putative representation of the SaCas9 variants.

### Results

### Validating MLDE Model for Predicting SpCas9's Activity

For protein engineering, it is challenging to investigate the vast combinatorial mutational space. Machine learning-based methods allow efficient exploration of the functional impact brought by mutations and breaking through the experimental limits of testing a great number of combinatorial mutants. The possibility for the ML-based in silico screen to be applied to the Cas9 optimization can be determined based on a small fraction of variants with experimentally determined activities from a combinatorial mutant library. In particular, using the previously published combinatorial mutagenesis data on SpCas9⁸, the minimal sample size sufficient for accurately predicting which variants possess top enzyme activities for the library can be readily determined.

In embodiments of the subject invention, a MLDE model that predicts activities of variants from multi-sites saturated mutagenesis libraries based on a small sample of variants is employed. The MLDE model offers numerous embeddings and models parameters, and the simple Georgiev embeddings²⁵ and the learnt embedding from Belper et al²⁶ are selected to combine with more complex neural networks models (parameter 1) or with an ensemble of more simple models such as random forests and SVM (parameter 2) to model the activities of SpCas9.

Different input sizes including 10%, 20%, 50%, 70% of randomly down-sampled empirical data points from the library of 650 variants are utilized as the training data for testing the SpCas9 activity. Since a previous study has showed that sampling diverse samples improves the ML performance²⁷, whether using a sample with high diversity may improve accuracy needs to be determined.

Deciding which characteristic of the data is most useful as the training data facilitates design of the library for building variants for empirical testing. To this end, more dissimilar variants are selected by reducing the numbers of variants sharing merely one and two sequence mismatches included in the input dataset.

In particular, when there are a limited number of input data points, for example, 10% and 20%, it is observed that restricting the number of one-mismatch and two-mismatches counterparts of each variant boosts the number of variants by, for example, 8% to 16%, harboring five to seven mismatches from each other in the dataset. When the sample size increases, such a selective scheme does not confer more dissimilarities among variants compared to the random selection. Overall, the diversity is preserved in the down-sampling.

The MLDE model is run on all the datasets to calculate the variables such as precision, specificity, and sensitivity for predicting variants with at least 70% of wild-type activity. Consistent with the little increase in diversity described above, it is found that diverse dataset generates slightly more variants with >70% of wild-type activity (i.e., greater sensitivity), but with a small compromise on higher false-positive discovery (i.e., lower precision and specificity), compared to the randomized selection as shown in FIG. 5. To purge false positives so as to reduce the burden of experimental validations, the randomized selection scheme that shows higher precision for the subsequent protein optimization is utilized.

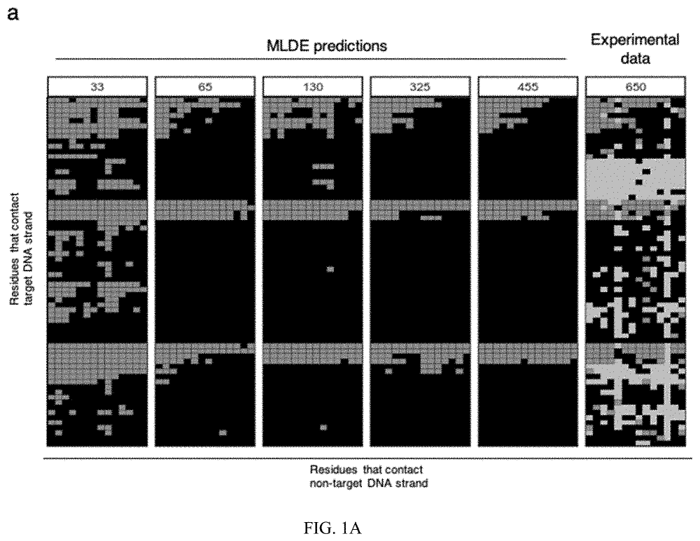

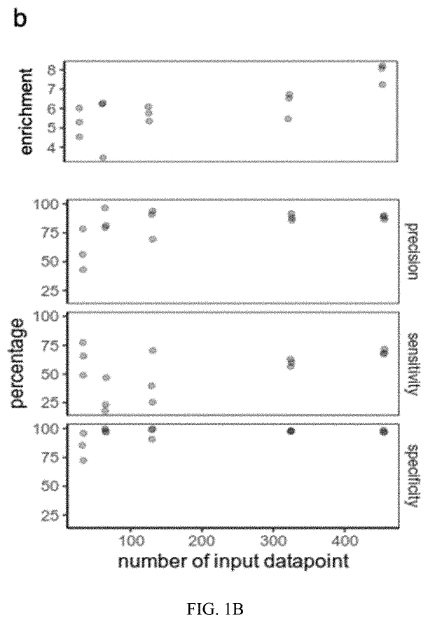

In the ML model runs, it is found that the prediction on SpCas9 activity achieves good precision and specificity as shown in FIG. 5. Using merely 10% of input is sufficient to identify the three clusters of variants with high activities, and consistent identification of variants with at least 70% of wild-type activity across 10%, 20%, 50%, and 70% of input is observed in FIG. 1A. It is also found that utilization of the Belper embedding and the model parameter 1 provides the best results, for example, average precision=87.3%, specificity=97.4%, and sensitivity=58.4%, as shown in FIG. 5. The high level of precision guarantees that the top-performing variants predicted by the MLDE model lead to a low level of false positives, thereby saving efforts in downstream experimental validations.

The Belper with parameter 1 configuration also exhibits high enrichment of functional variants among the top 5% hits in the prediction. With 10% and 20% of input, 81.6% and 85.2% of variants are functional among the top 5% hits from the predictions, which correspond to a 5.46-fold and 5.88-fold enrichment of finding a functional variant compared to the null background, respectively, as shown in FIG. 1B. Taking into account both precision and enrichment, it is determined that 20% of input can be used as the input threshold that achieves relatively robust and consistent performance as shown in FIG. 1B. Moreover, 10% of input can be used to further reduce the experimental screening burden with enrichment and sensitivity slightly being compromised as shown in FIG. 1B.

Therefore, functional variants with high on-target activities can be readily isolated in silico based on the MLDE model with Belper embedding and modelling parameter 1, when empirical measurements of, for example, 10-20%, of variants are provided as input.

### Experimentally Validated MLDE Prediction Identifies Activity-Enhanced KKH-SaCas9 Variants

Based on the parameters that yield a good prediction of the SpCas9's on-target activity, the MLDE model can be applied for optimization of the SaCas9. The test results show that the editing activity of KKH-SaCas9 is augmented, suggesting that introducing additional non-base-specific interactions between KKH-SaCas9 and the PAM duplex of the target DNA can increase the efficiency of the enzyme. Such strategy is effective in compensating the reduced DNA base-specific interactions of an engineered SpCas9 variant that broaden its PAM compatibility and restoring the enzyme's activity²⁸. For SaCas9, Nishimasu et al. has illustrated in the crystal structure (5CZZ) its amino acid residues that show direct contact with the target DNA backbone of the PAM duplex¹⁷.

In one embodiment, eight amino acid residues (located within the WED and PI domains of KKH-SaCas9) that interact with and surround the PAM duplex for combinatorial mutagenesis are selected as shown in Table 5 below. Based on a rational design, up to three amino-acid alternatives to the wild-type residue are selected for each site, leading to a total of 1,296 variant combinations.

TABLE 5
Amino acid residues selected for mutagenesis

| Amino acid | Domain | Reason for selection | | |
|---|---|---|---|---|
| residue(s) | location | in this study | Mutation(s) | Reference |
| L887 | WED | Close proximity to | Arginine has the length | Nishimasu et al., |
| | | N888 and A889, | and best capacity to | Cell, 2015 |
| | | substitutions may help | potentially interact in | (PMID: |
| | | increase stability of | different | 26317473) |
| | | protein. | conformations. | |
| N888 | WED | Interaction with | Glutamine is longer in | Nishimasu et al., |
| | | backbone of PAM | structure than | Cell, 2015 |
| | | duplex | asparagine that could | (PMID: |
| | | | have better access to | 26317473) |
| | | | the backbone. | |
| A889 | WED | | Arginine could provide | Tan et al., PNAS, |
| | | | additional electrostatic | 2019 (PMID: |
| | | | charges for stronger | 31570596), |
| | | | interaction with | Luscombe et al., |
| | | | backbone and serine | NAR, 2001 |
| | | | was said to contribute | (PMID: |
| | | | to majority of bonds | 11433033) |
| | | | with DNA backbone, | |
| | | | mainly providing | |
| | | | stability. | |
| N985 | PI | Direct contact with | Aspartate and leucine | Nishimasu et al., |
| | | 4/5th base of PAM | may have less | Cell, 2015 |
| | | | interactions with the | (PMID: |
| | | | PAM. | 26317473) |
| N986 | PI | Interaction with the 5th | Threonine being in the | Nishimasu et al., |
| | | base of PAM | same group as | Cell, 2015 |
| | | | asparagine but having a | (PMID: |
| | | | shorter structure could | 26317473) |
| | | | help reduce the amount | |
| | | | of interactions with | |
| | | | PAM. Leucine takes on | |
| | | | a similar structure to | |
| | | | asparagine, and may | |
| | | | prevent interactions | |
| | | | with the 5th position | |
| | | | while increasing | |
| | | | flexibility by reducing | |
| | | | the amount of | |
| | | | interactions with the | |
| | | | surrounding residues. | |
| L988 | PI | Mutations were reported | Aspartic acid could | Nishimasu et al., |
| | | to reduce PAM | provide some repulsion | Cell, 2015 |
| | | constraint at the 5th | and was also used in | (PMID: |
| | | base of PAM | previous study to | 26317473), Ma et |
| | | | reduce binding to | al., Nature Com, |
| | | | PAM. | 2019 (PMID: |
| | | | | 30718489) |
| L989 | PI | Decrease interactions | Arginine was used in | Ma et al., Nature |
| | | with the residues | previous study which | Com, 2019 |
| | | involved in binding to | showed reduced | (PMID: |
| | | PAM | interactions with PAM. | 30718489) |
| R991 | PI | Reported changes in this | Glutamine has a long | Nishimasu et al., |
| | | position could help | structure but less | Cell, 2015 |
| | | reduce PAM | electrostatic to | (PMID: |
| | | constraints, interacts | arginine, and isoleucine | 26317473), Ma et |
| | | with 4th, 5th and 6th | for non-base specific | al., Nature Com, |
| | | PAM bases. | interactions | 2019 (PMID: |
| | | | | 30718489) |

Moreover, 300 out of the 1,296 (23%) variants are randomly picked, generating empirical data from a screening library as the training set input, and the MLDE model is run with the Belper embedding and the modelling parameter 1 to predict functional variants that have activities comparable to wild-type, for example, at least 70%, from the full variant space. The generated in silico prediction results are then confirmed by the experimental screening data, validating that the MLDE model predicts KKH-SaCas9's activity with high accuracy.

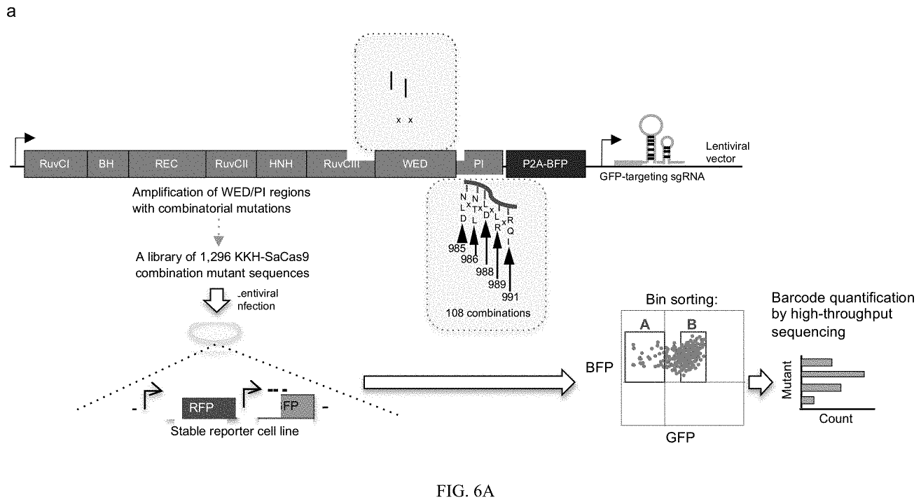

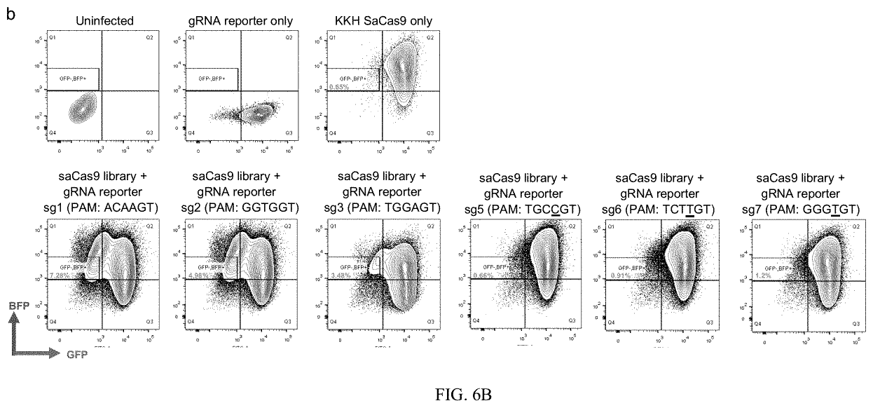

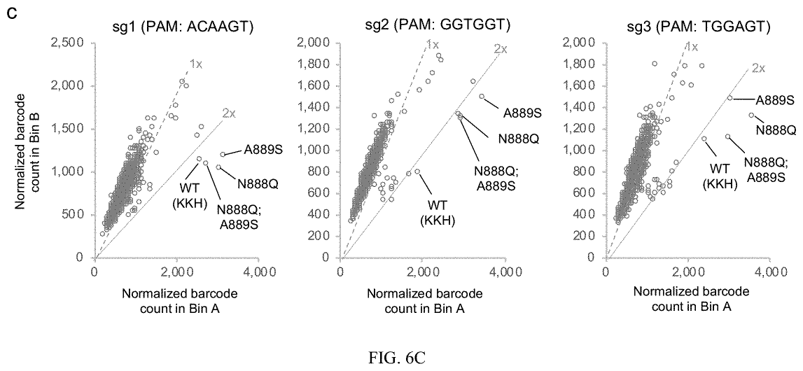

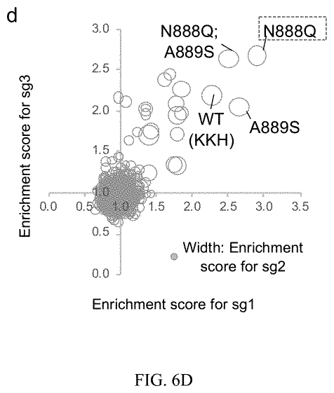

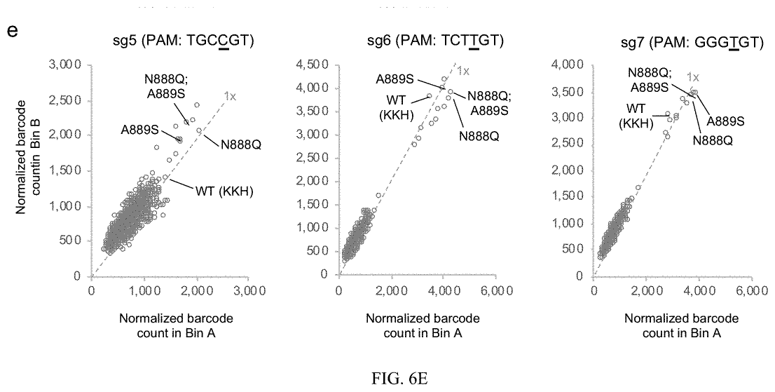

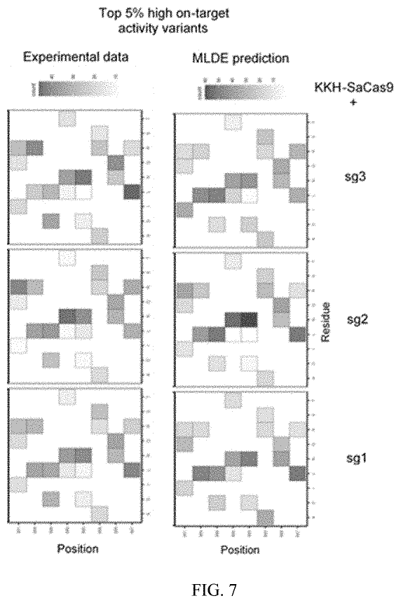

In one embodiment, a full-coverage screening library of 1,296 variants is assembled and the library is delivered by lentiviruses into reporter cell lines that stably expressed GFP and a sgRNA targeting the GFP gene sequence as shown in FIG. 6A. Variants generate indel-mediated disruption of the GFP sequence and its expression is enriched in the sorted bin with low GFP fluorescence (i.e., Bin A) as compared to the GFP-positive population (i.e., Bin B) as shown in FIGS. 6A and 6B. The mutated sequences on KKH-SaCas9 are retrieved using Illumina NovaSeq and the activities for the library of KKH-SaCas9 variants are plotted based on their relative enrichment in the sorted bins for example as shown in FIG. 6C.

The experimental screening results reveal that variants harboring mutations at residues 888 and 889 of the WED domain and 988 and 989 of the PI domain are frequently detected among the top 5%-ranked variants with high on-target activities, while those carrying wild-type sequences at 887 of the WED domain and 985, 986, and 991 of the PI domain more likely confer the enzyme with higher activity as shown in FIG. 7. Based on the library of the variants, it is identified that two of them, harboring N888Q and N888Q/A889S, exhibit activity higher than the KKH-SaCas9, when paired with 2 out of 3 tested sgRNAs (i.e., sg1 and sg3). For the third sgRNA (i.e., sg2), the two variants show editing efficiency comparable to that of the KKH-SaCas9 as shown in FIGS. 6C and 6D. When employing other 3 sgRNAs targeting the GFP sequence harboring non-permissive PAMs for KKH-SaCas9 (i.e., NNNYRT), the library variants including the N888Q and N888Q/A889S variants show minimal effects on disrupting GFP expression, indicating that the variants do not have relaxed constraints at those PAMs for example as shown in FIG. 6E.

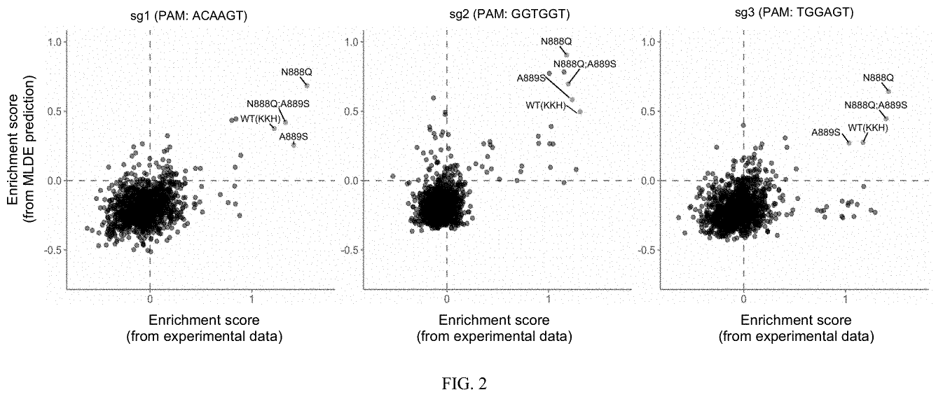

Comparison between the in silico prediction results and experimental screen data indicates that the MLDE model accurately predicts KHH-SaCas9's activity. It is found that the three independent sets of activity measurements on KKH-SaCas9 variants yield predictions consistent with the experimental screen data, for example those as shown in FIG. 2. Among them, the variant N888Q is also predicted by the MLDE model as the top-performing variants of all three sgRNAs as shown in FIG. 2. High similarity is observed in comparison of variants with the top 5% predicted activities overall as shown in FIG. 7. These results are in agreement with the SpCas9 activity prediction, demonstrating that the MLDE model can identify top-performing variants at a low false-positive rate (i.e., high precision). The high level of consistency, including the identification of the same top-performing variants, between the in silico and experimental screen data, confirms that the MLDE model is effective for predicting the activity of the KKH-SaCas9.

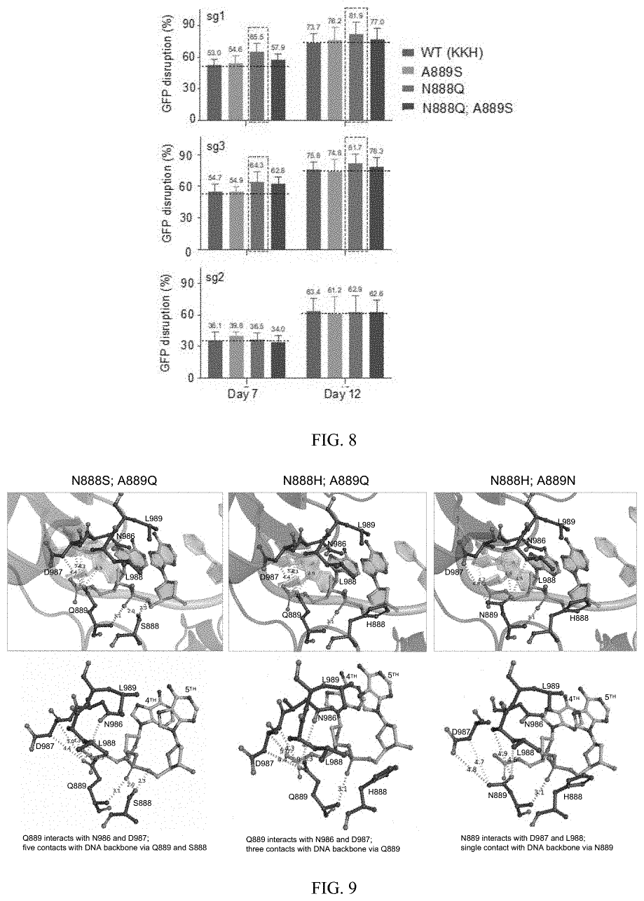

To further verify the editing efficiencies of the identified variants with increased KKH-SaCas9's activity, individual validation assays are performed. The validation results are consistent with the screening data, revealing that the N888Q and N888Q/A889S variants exhibit increased editing activities over KKH-SaCas9, when paired with sg1 and sg3 sgRNAs as shown in FIG. 8. As a result, the screen identifies residues located proximal to the PAM duplex that can be modified to increase KKH-SaCas9's on-target activity.

### Structure-Guided Engineering of Activity-Enhanced KKH-SaCas9-Plus

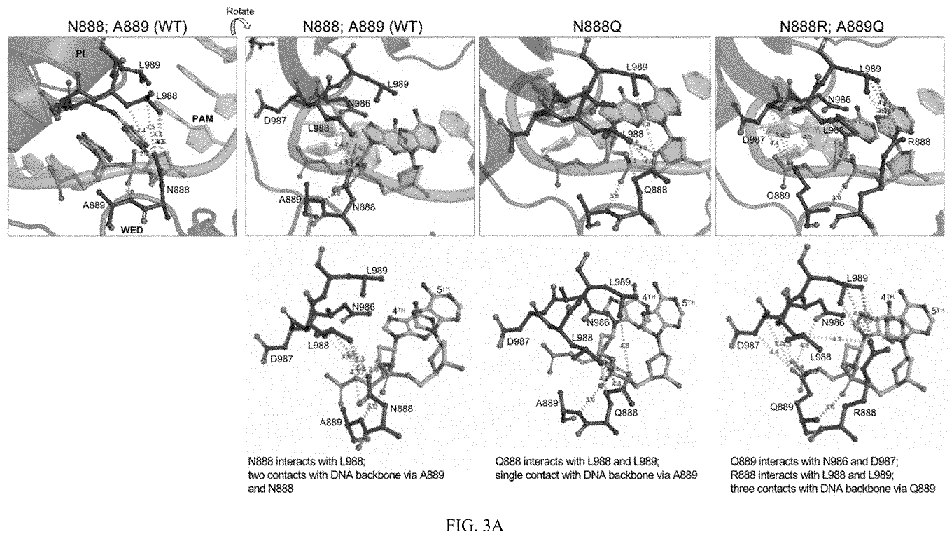

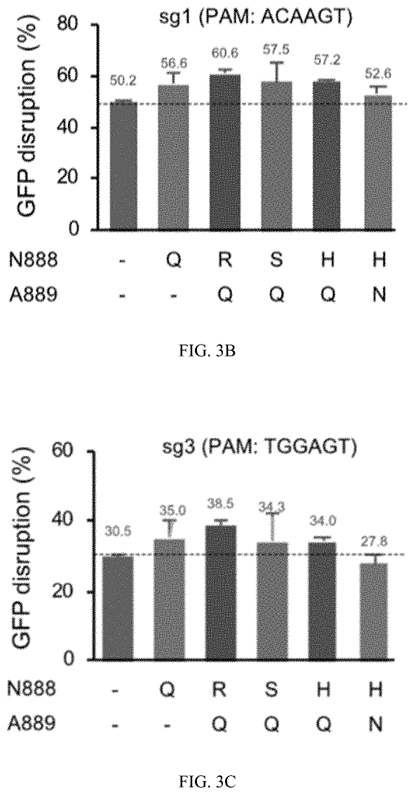

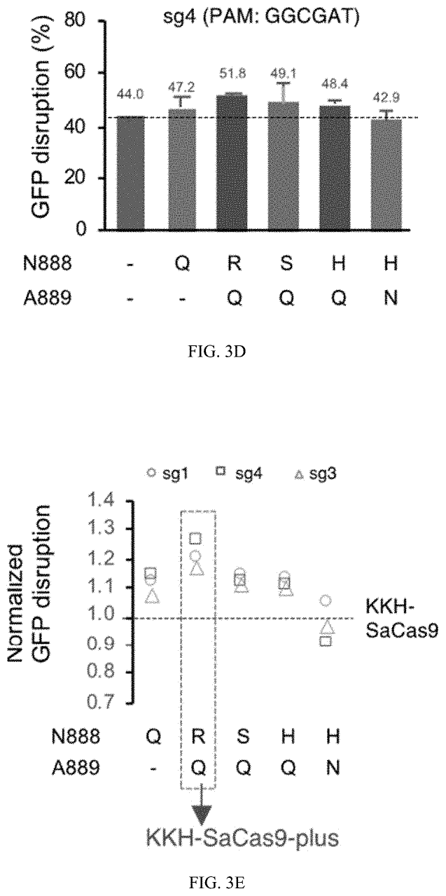

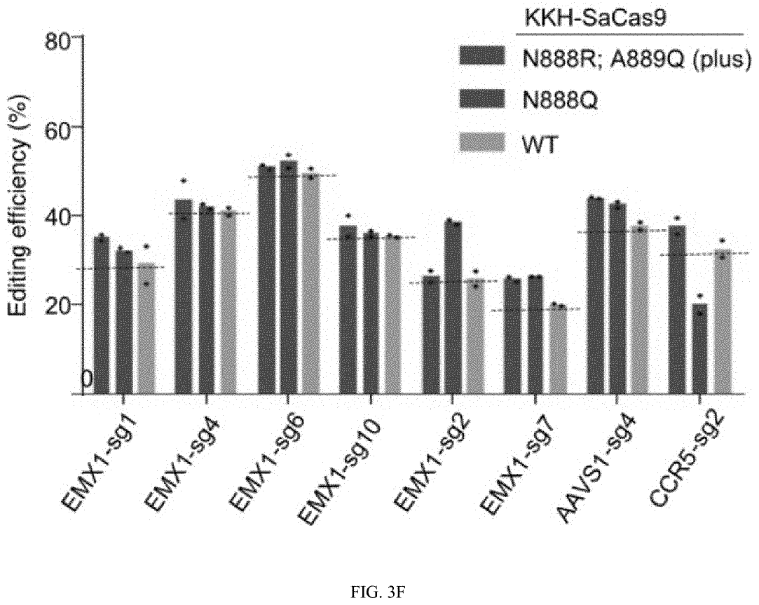

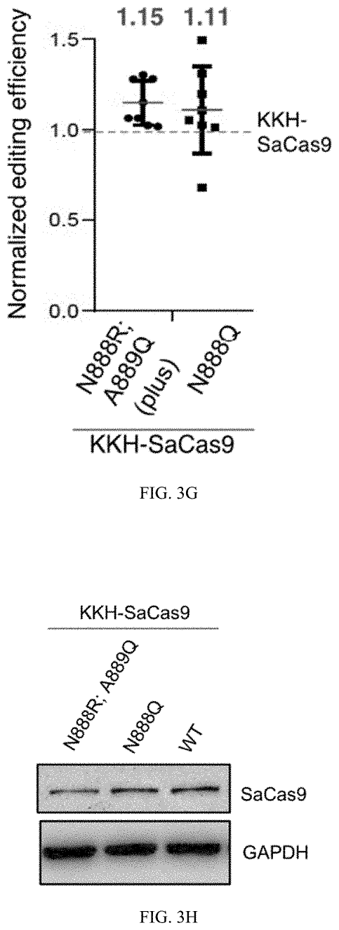

Based on the above identified activity-enhanced variants, structure-guided engineering is employed to further improve the editing activity of KKH-SaCas9. Protein structure analyses indicate that N888 and A889 at the WED domain of SaCas9 are positioned close to its PI domain and the DNA backbone of the PAM duplex¹⁷. Previous modelling also revealed that while N888Q removes its contact with the DNA backbone of the PAM duplex, it could increase its proximity to and add interactions with L989 at the PI domain as shown in FIG. 3A. The interactions may sandwich the PAM duplex more firmly to facilitate unwinding of the target DNA and trigger base pairing between the sgRNA and the DNA target, enabling greater editing activity for the N888Q and N888Q/A889S variants.

In one embodiment, tests are performed to confirm that switching N888 and A889 to other residues could strengthen the interactions between WED and PI domains and also enhance KKH-SaCas9's activity. Four more combined mutation variants are engineered on these positions, which are selected based on predicted contact gains with the PI domain via N986, D987, L988, and/or L989 as shown in FIG. 3A and FIG. 9. Three variants, namely, N888H/A889Q, N888S/A889Q, and N888R/A889Q, that exhibit activity greater than KKH-SaCas9 carry a common A889Q mutation, while the fourth variant that contains A889N instead of A889Q (i.e., N888H/A889N) shows activity comparable to KKH-SaCas9 as shown in FIGS. 3B-3E. The result suggests that A889Q increases the editing activity of KKH-SaCas9. Further, the modelling shows that putatively N888Q only adds contact with the PI domain via L989. However, A889Q is predicted to interact with N986 and D987, as well as adding contacts with the DNA backbone of the PAM duplex as shown in FIG. 3A.

Among the variants tested, the one harboring N888R/A889Q mutations (hereafter designated as “KKH-SaCas9-plus”) exhibit the greatest editing activity, for example, 122% of the activity of KKH-SaCas9 averaged from 3 sgRNAs targeting GFP as shown in FIGS. 3B-3E. It is further confirmed that KKH-SaCas9-plus generates more edits when targeting endogenous genes. For example, 115% of the activity KKH-SaCas9 averaged from sgRNAs targeting 8 loci is shown in FIGS. 3F-3G, while 3 out of the 8 loci have as much as 30% enhancement of the editing activity. The N888Q variant shows an average on-target editing activity of 111% for KKH-SaCas9 at these endogenous loci as shown in FIGS. 3F-3G. Referring to FIG. 3H, it is verified that the increase of editing activities is not due to the difference in the variants' protein expression.

Moreover, the modelling of KKH-SaCas9-plus shows that it contacts the PI domain via N986, D987, L988, and/or L989 residues and has three contacts with the DNA backbone as shown in FIG. 3A. Whereas, the relatively fewer activity-enhanced variants carrying N888H/A889Q and N888S/A889Q mutations could interact with the PI domain only via N986/987, but not L988/L989, with an equal number of or more contacts with the DNA backbone as shown in FIG. 9. Hence, the creation of new interactions between the WED and PI domains at multiple locations within the PAM duplex region may be effective in enhancing the KKH-SaCas9's activity, accounting for the greater enhancement for KKH-SaCas9-plus.

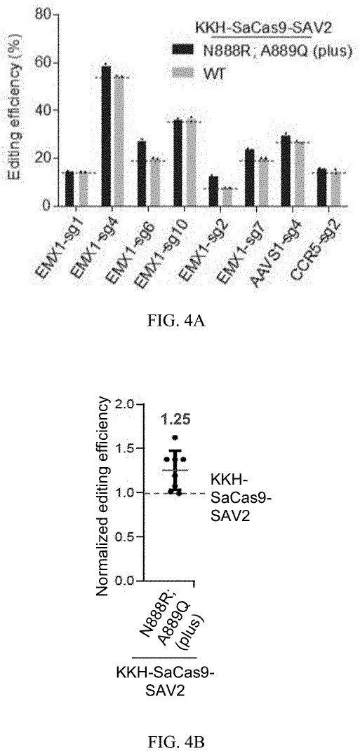

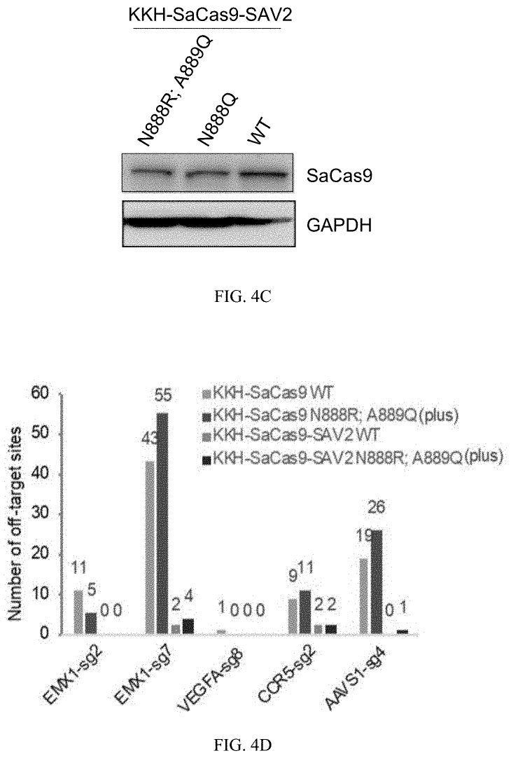

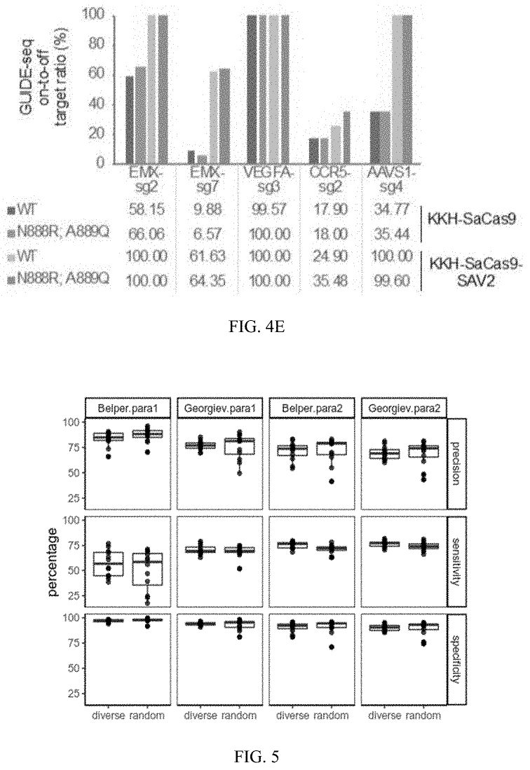

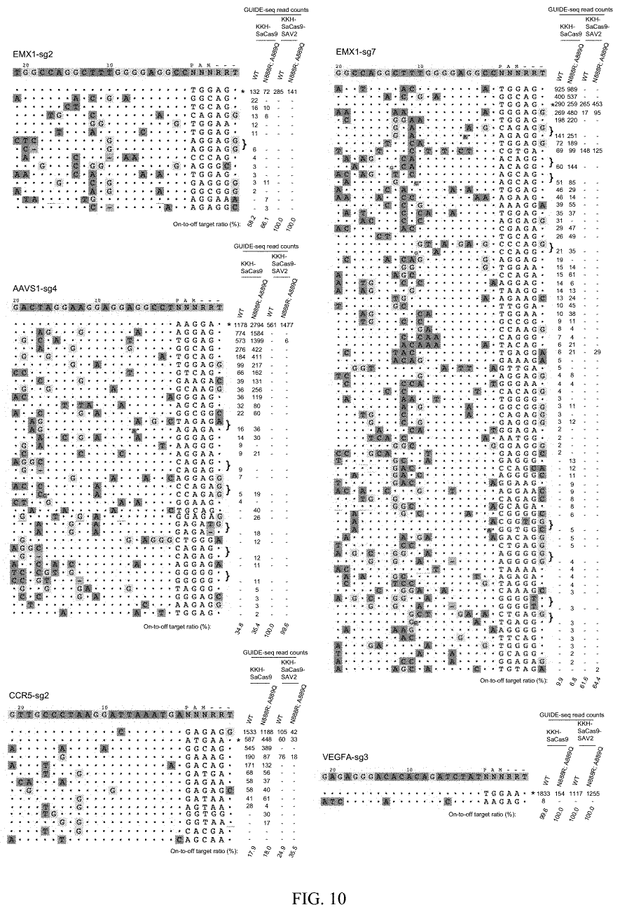

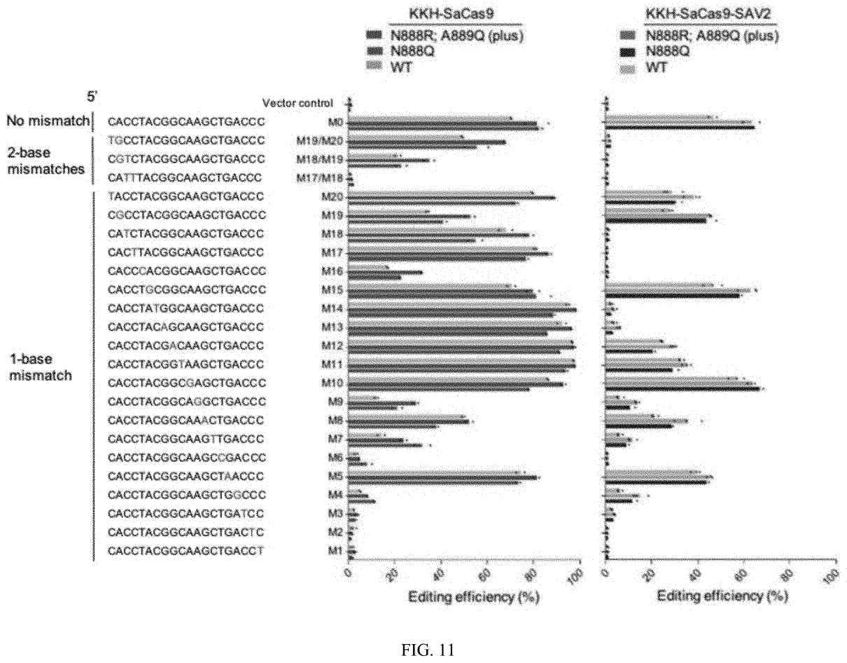

It is determined that the addition of N888R/A889Q can improve the activity of high-fidelity variants of KKH-SaCas9, such as the newly engineered SAV2. Moreover, it is found that the N888R/A889Q enhances the on-target activity of SAV2. For example, 125% of KKH-SaCas9's activity averaged from sgRNAs targeting 8 loci is observed as shown in FIGS. 4A-4C. Notably, as revealed by the GUIDE-seq, the mutation-combined variant, for example, KKH-SaCas9-SAV2-plus, generates much reduced genome-wide off-target editing, and its level is comparable with SAV2 as shown in FIGS. 4D-4E and FIG. 10. This variant is able to discriminate all three tested two-base pairs and many of the single-base pair mismatches that span over the entire protospacer sequence, while exhibiting increased on-target activity as shown in FIG. 11. These results indicate feasibility to combine activity-enhancing and specificity-enhancing mutations for enhancing the enzyme's from on-target activity to off-target activity.

There have been tremendous efforts in designing Cas9 proteins to boost gene editing efficiency and purge undesired off-target editing at the same time by maintaining a delicate balance between interacting and non-interacting amino-acid side chains of the Cas9 protein with the sgRNA-DNA complex. Dozens of variants possessing different mutation combinations have been reported thus far, each representing one of the many optimal solutions for the trade-off between Cas9 activity and precision.

Considering that any of the amino-acid sites of SaCas9 in spatial proximity to the sgRNA-DNA complex are potential sites for optimization, which could reach as many as 40 sites¹⁷, the number of combinatorial variants, for example, 2⁴⁰=1.1×10¹², to screen through for optimization is prohibitively high for wet-lab experiments, even if each site is restricted to two (wild-type or mutated) amino-acid residues.

Previous studies have shown that with a rational design, each site can be limited to 4-5 candidate residues and that a targeted mutagenesis library can be generated to reduce screening efforts. SpCas9 variants with both high activity and fidelity have been successfully identified from a combinatorial screen of 952 variants⁸.

In embodiments of subject invention, a rational design-based screen with machine learning is adopted for optimization of the Cas9 proteins. Particularly, the ability of ML to further downsize the experimental screen via the extrapolation of handfuls of variants with experimentally-determined fitness values is assessed. It is found that ML-based in silico screen greatly facilitates the search of more efficient Cas9 variants. In the ML runs on the SpCas9 dataset using as little as 10% of variants as input training data, a 81.6% chance of capturing functional variants among the top 5% of variants predicted is achieved. Shortlisting a few candidate residues on selected amino-acid sites via structure-guided rational design of SpCas9 significantly enhances the chances of finding better variants from the previously published combinatorial mutant library. Similarly, the results of the MLDE model suggest that focus should be placed on surveying diverse sequence spaces deemed to contain functional variant²⁴. In an independent Cas9 optimization task, it is further demonstrated that the MLDE model exhibits surpassing performance in the prediction of KKH-SaCas9 variants' activities on three sgRNAs and shows success in identifying useful novel variants in the KKH-SaCas9 screen subsequently. When the combined approach of structure-guided design, targeted mutagenesis library screen, and ML is employed to identify activity-enhanced KKH-SaCas9 variants, the path to identify the top variants is significantly shortened.

The best-performing variant, KKH-SaCas9-plus, harbors N888R/A889Q mutations, improving its editing activity. The molecular modelling provides structural insights that these mutations may strengthen the interactions between KKH-SaCas9's WED and PI domains located near the PAM duplex to anchor the target DNA in the SaCas9-sgRNA-target DNA complex. While N888R/A889Q increases the on-target activity, the mutations only minimally affect the off-target activity of SAV2 which is a high-fidelity derivative of KKH-SaCas9. The result affirms that the abilities of KKH-SaCas9 to bind the DNA and distinguish base mismatches between sgRNA and the DNA target probably act through distinct mechanisms, and thus its activity and specificity could be engineered independently. It is possible that N888R/A889Q is also compatible with other dSaCas9-derived genome perturbation tools including gene activators^{31,32}, base editor^{33,34} and prime editor³⁵ to increase their abilities to bind the DNA and thus their activities. The N888R/A889Q mutations on the WED domain represent a useful building block for further engineering of various genome perturbation tools to achieve both high activity and high specificity.

To discover the activity-enhanced KKH-SaCas9 variants, a smaller pool of, for example, about a thousand variants are initially experimented based on the structure-guided design. It is noted that the selection of suitable sgRNAs, for example, sg50N for SpCas9 and sg1, sg2, and s3 for KKH-SaCas9, allows the MLDE model to generate more reliable predictions in subsequent screens. The MLDE-based workflow is tested and validated based on the experimental screening data and the required number and diversity of the input combinations are defined for in silico predictions. The results lead to screening of more combinatorial mutations by creation of a directed library on a manageable experimental scale. Continual efforts in advancing ML methods for protein structure modelling, including incorporating structural descriptors³⁶ into the learnt representation, lead to improvement of the prediction on variants' activities for in-silico screens.

Nevertheless, only mutation combinations from selected amino acid residues by a rational design are investigated, without exploration of the performance of the MLDE model on a virtual fully saturated mutagenesis screen. Creating a more comprehensive screening strategy by designing a library enriched with diverse but not “dead” variants remains challenging. One could examine possible structural changes of the designed variants predicted using other in silico tools such as DynaMut³⁷, Rosetta^{38,39}, and Pymol to further filter for candidate mutations. For example, experimental screening of a computationally designed library of ubiquitin variants was shown to be successful in identifying variants with strong protein-binding ability⁴⁰.

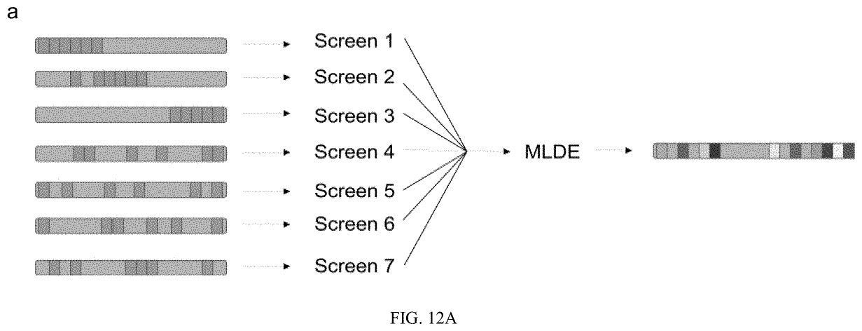

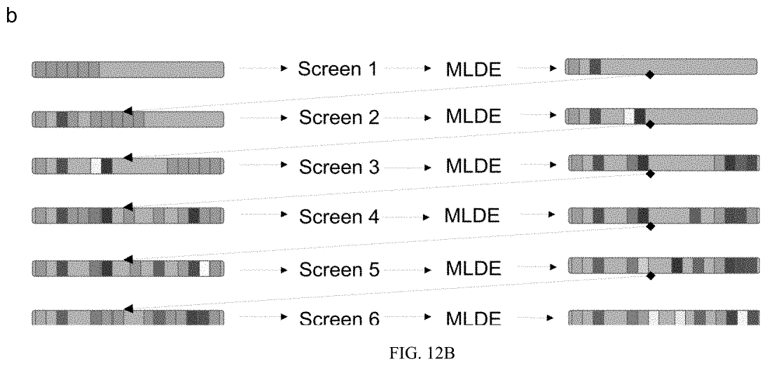

Moreover, increasing the number of amino-acid sites is desirable. It would be particularly useful for protein repurposing to use another substrate, where the wild-type has essentially no activity. For example, obtaining a “PAMless” SaCas9 involves engineering multiple sites beyond the PI and WED domains. The number of targeted mutagenesis sites to be incorporated is still a confounding factor in combinatorial library construction. For example, commercial oligo synthesis of a 100 bp DNA fragment at most accommodates 10 sites of NNN/NNK degenerate codons or trinucleotide pool. Thus, the MLDE model is advantageous in transcending such physical limitations by building a combined in silico screen supplied with empirical data from multiple smaller focused libraries. For example, multiple focused screens may be performed with MLDE converging sites with modest overlaps that each library has mutagenesis of 5 amino-acid residues per site, up to 6 sites with 1-2 sites in common to another library as shown in FIG. 12A. Further, the MLDE model can be used to combine all these experimental data in silico to predict the optimal variants. Alternatively, iterative rounds of targeted mutagenesis can be performed as shown in FIG. 12B. The best variants found at the end of each round seed the mutagenesis library of the next round with a new set of amino-acid sites subjected to mutagenesis. In both screening schemes, MLDE model and other ML-based methods play an important role in the search for high-performance variant and serve as an invaluable tool in the toolkit of protein engineering. Complementary methods, including polymerase chain reaction-based mutagenesis and CombiSEAL^{8,41} that allow assembly of combinatorial mutations scattered over the entire protein can facilitate building and experimenting the desirable targeted mutagenesis libraries.

Comparison of MLDE Performance on Predicting SpCas9 Activity with That on Predicting sg50N and sg80N sgRNAs

The performance of the MLDE model on surveying the SpCas9's activity are compared with the performance of the MLDE model on surveying data of two sgRNAs⁸, namely, sg50N (650 empirical datapoints) and sg8ON (729 empirical datapoints), that target on a red fluorescent protein (RFP) sequence as the input data.

Similar to the approach adopted for testing sg50N describe below, input datasets including 10%, 20%, 50%, and 70% of empirical measurements are generated to test the minimal input for effective selection of top variants from the MLDE prediction, corresponding to datasets of 73, 146, 365, and 510 empirically measured on-target activity for sg8ON.

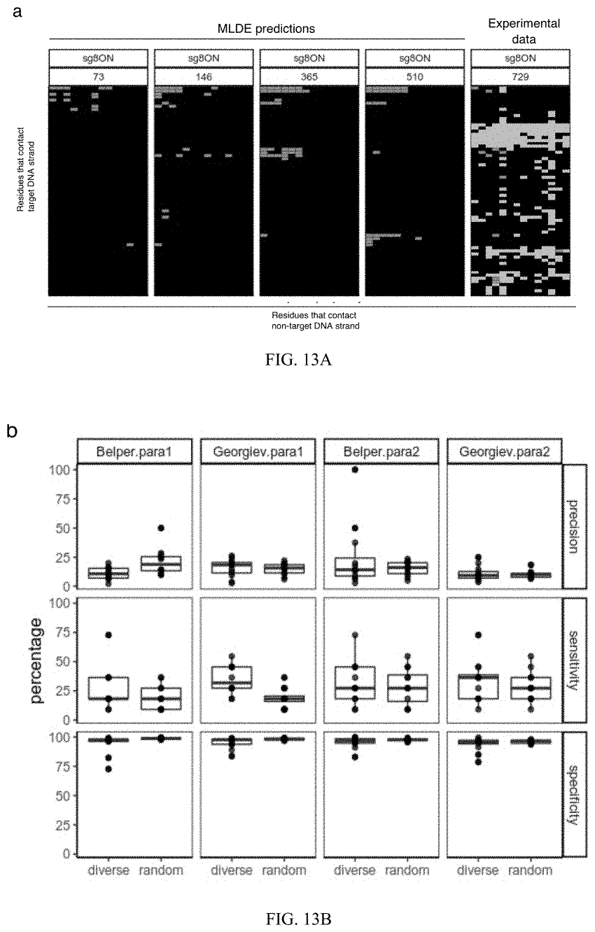

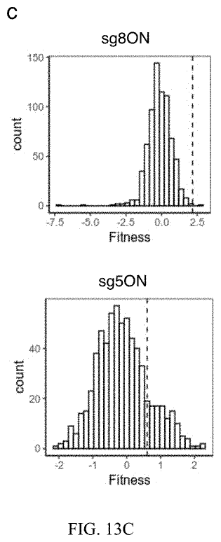

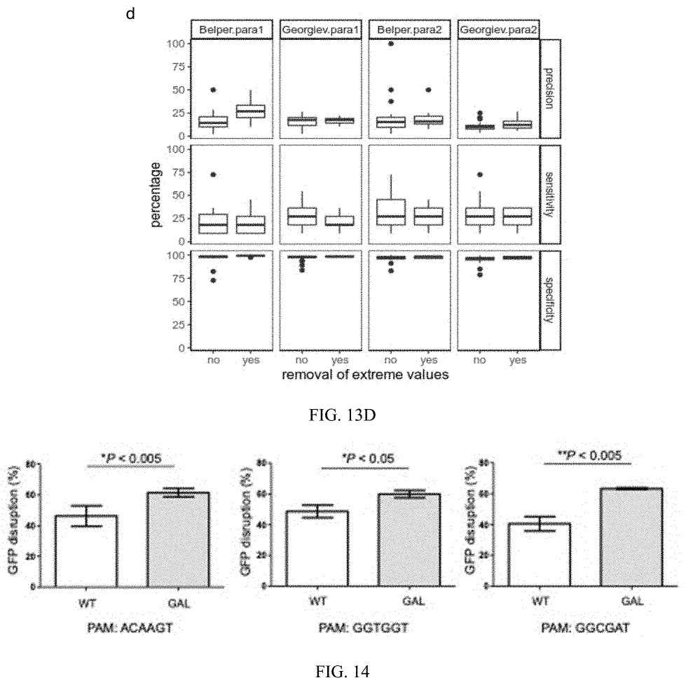

When the datasets of the two sgRNAs are compared, it is found that the prediction on sg50N activity achieves precision and specificity that are higher than these of the sg8ON activity. While using merely 10% input is sufficient to identify the three clusters of variants with high sg50N activity as shown in FIG. 1A, using 50% input does not reliably identify top variants with high sg8ON activity as shown in FIG. 13A.

Accordingly, the performance of the MLDE model on sg8ON is much lower than that on sg50N as shown in FIG. 13B. The overabundance of variants show smaller than 70% of the wild-type activity in the sg8ON dataset (only 11 variants show >=70% of wild-type activity among 792 experimentally tested variants) as shown in FIG. 13A. On average, merely two out of eleven variants are uncovered to show >=70% of wild-type activity in the sg8ON datasets, regardless of the size of input training data. The rarity of variants with >=70% of wild-type activity in the sg8ON dataset inhibit the learning capability of the ML model. In addition, the sg8ON activities have a narrow range (5%-95% of data range=0.58-0.83) compared to the distribution of sg50N activities (5%-95% of data range=0.18-0.78) as shown in FIG. 13C, making the training of the MLDE model more difficult. Setting a floor activity threshold, for example, assigning −3 to the four variants with an enrichment score lower than 3 before min-max normalization to expand the data range (5-95% of data have range=0.29-0.71), only results in modest improvement in the precision as shown in FIG. 13D.

Thus, sg8ON is a challenging dataset for ML models. Nonetheless, the MLDE model exhibits surpassing performance in the prediction of SpCas9 variants on sg50N activities and shows success in identifying useful novel variants in the KKH-SaCas9 screen.

When facing such a phenomenon resulting from sgRNA-specific effect, the MLDE model may be limited in applications for identifying variants with improved performance. It is also observed in previous studies that some sgRNAs may be more susceptible to losing editing activity with a reducing functional dose of Cas9 (or Cas9:sgRNA molar ratio) used^{2,3}. Since the reasons accounting for such sgRNA-specific effect are not yet known, it may be desirable to test multiple conditions (i.e., more sgRNAs) and select, for example, sg50N for SpCas9 and sg1, sg2, and s3 for KKH-SaCas9, allowing the MLDE model to generate reliable predictions in subsequent screens.

In embodiments of the subject invention, the genome-editing Cas9 protein uses multiple amino-acid residues on its sequence to bind the target DNA. Considering only the residues in proximity to the target DNA as potential sites to optimize Cas9's activity, the number of combinatorial variants to screen through is too massive for a wet-lab experiment. It is demonstrated that a machine learning-coupled combinatorial mutagenesis approach reduces the experimental screening burden by as high as 90%, while achieving 87% prediction precision and 97% specificity, for Cas9 engineering. Using this approach, mutations that enhance the editing activity of the protospacer adjacent motif-relaxed KKH variant of Cas9 nuclease from Staphylococcus aureus (KHH-SaCas9) are discovered. The mutations located at SaCas9's WED domain are modelled to strengthen contacts with the PI domain and sandwich the protospacer adjacent motif-proximal DNA duplex. Followed by structure-guided engineering, one of the variants, named KKH-SaCas9-plus, showed as high as 30% enhancement of editing activity at multiple loci without compromising high genome-wide targeting specificity, when combined with mutations that confer KKH-SaCas9 with high accuracy. In addition to generating a KKH-SaCas9 nuclease with efficiency exceeding its wild-type counterpart, a readily applicable workflow is established, leveraging on the machine learning-assisted paradigm to accelerate engineering of genome editors.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1 Kleinstiver, B. P. et al. High-fidelity CRISPR-Cas9 nucleases with no detectable genome-wide off-target effects. Nature 529, 490-495, doi:10.1038/nature16526 (2016).
2 Slaymaker, I. M. et al. Rationally engineered Cas9 nucleases with improved specificity. Science 351, 84-88, doi:10.1126/science.aad5227 (2016).
3 Hu, J. H. et al. Evolved Cas9 variants with broad PAM compatibility and high DNA specificity. Nature 556, 57-63, doi:10.1038/nature26155 (2018).
4 Nishimasu, H. et al. Engineered CRISPR-Cas9 nuclease with expanded targeting space. Science 361, 1259-1262, doi:10.1126/science.aas9129 (2018).
5 Kleinstiver, B. P. et al. Engineered CRISPR-Cas9 nucleases with altered PAM specificities. Nature 523, 481-485, doi:10.1038/nature14592 (2015).
6 Casini, A. et al. A highly specific SpCas9 variant is identified by in vivo screening in yeast. Nat Biotechnol, doi:10.1038/nbt.4066 (2018).
7 Chen, J. S. et al. Enhanced proofreading governs CRISPR-Cas9 targeting accuracy. Nature 550, 407-410, doi:10.1038/nature24268 (2017).
8 Choi, G. C. G. et al. Combinatorial mutagenesis en masse optimizes the genome editing activities of SpCas9. Nat Methods 16, 722-730, doi:10.1038/s41592-019-0473-0 (2019).
9 Lee, J. K. et al. Directed evolution of CRISPR-Cas9 to increase its specificity. Nat Commun 9, 3048, doi:10.1038/s41467-018-05477-x (2018).
10 Vakulskas, C. A. et al. A high-fidelity Cas9 mutant delivered as a ribonucleoprotein complex enables efficient gene editing in human hematopoietic stem and progenitor cells. Nat Med 24, 1216-1224, doi:10.1038/s41591-018-0137-0 (2018).
11 Ran, F. A. et al. In vivo genome editing using Staphylococcus aureus Cas9. Nature 520, 186-191, doi:10.1038/nature14299 (2015).
12 Tan, Y. et al. Rationally engineered Staphylococcus aureus Cas9 nucleases with high genome-wide specificity. Proc Natl Acad Sci USA 116, 20969-20976, doi:10.1073/pnas.1906843116 (2019).
13 Kleinstiver, B. P. et al. Broadening the targeting range of Staphylococcus aureus CRISPR-Cas9 by modifying PAM recognition. Nature biotechnology 33, 1293-1298, doi:10.1038/nbt.3404 (2015).
14 Ma, D. et al. Engineer chimeric Cas9 to expand PAM recognition based on evolutionary information. Nature Communications 10, 560, doi:10.1038/s41467-019-08395-8 (2019).
15 Luan, B., Xu, G., Feng, M., Cong, L. & Zhou, R. Combined Computational-Experimental Approach to Explore the Molecular Mechanism of SaCas9 with a Broadened DNA Targeting Range. J Am Chem Soc 141, 6545-6552, doi:10.1021/jacs.8b13144 (2019).
16 Nishimasu, H. et al. Crystal structure of Cas9 in complex with guide RNA and target DNA. Cell 156, 935-949, doi:10.1016/j.cell.2014.02.001 (2014).
17 Nishimasu, H. et al. Crystal Structure of Staphylococcus aureus Cas9. Cell 162, 1113-1126, doi:10.1016/j.cell.2015.08.007 (2015).
18 Yang, K. K., Wu, Z. & Arnold, F. H. Machine-learning-guided directed evolution for protein engineering. Nat Methods 16, 687-694, doi:10.1038/s41592-019-0496-6 (2019).
19 Bedbrook, C. N. et al. Machine learning-guided channelrhodopsin engineering enables minimally invasive optogenetics. Nat Methods 16, 1176-1184, doi:10.1038/s41592-019-0583-8 (2019).
20 Mason, D. M. et al. Optimization of therapeutic antibodies by predicting antigen specificity from antibody sequence via deep learning. Nat Biomed Eng 5, 600-612, doi:10.1038/s41551-021-00699-9 (2021).
21 Bryant, D. H. et al. Deep diversification of an AAV capsid protein by machine learning. Nat Biotechnol 39, 691-696, doi:10.1038/s41587-020-00793-4 (2021).
22 Biswas, S., Khimulya, G., Alley, E. C., Esvelt, K. M. & Church, G. M. Low-N protein engineering with data-efficient deep learning. Nat Methods 18, 389-396, doi:10.1038/s41592-021-01100-y (2021).
23 Wu, Z., Kan, S. B. J., Lewis, R. D., Wittmann, B. J. & Arnold, F. H. Machine learning-assisted directed protein evolution with combinatorial libraries. Proc Natl Acad Sci USA 116, 8852-8858, doi:10.1073/pnas.1901979116 (2019).
24 Wittmann, B. J., Yue, Y. & Arnold, F. H. Informed training set design enables efficient machine learning-assisted directed protein evolution. Cell Syst, doi:10.1016/j.cels.2021.07.008 (2021).
25 Georgiev, A. G. Interpretable numerical descriptors of amino acid space. J Comput Biol 16, 703-723, doi:10.1089/cmb.2008.0173 (2009).
26 Bepler, T. & Berger, B. Learning protein sequence embeddings using information from structure International Conference on Learning Representations, doi:arXiv:1902.08661v2 (2019).
27 Romero, P. A., Krause, A. & Arnold, F. H. Navigating the protein fitness landscape with Gaussian processes. Proc Natl Acad Sci USA 110, E193-201, doi:10.1073/pnas.1215251110 (2013).
28 Hirano, S., Nishimasu, H., Ishitani, R. & Nureki, O. Structural Basis for the Altered PAM Specificities of Engineered CRISPR-Cas9. Molecular Cell 61, 886-894, doi:https://doi.org/10.1016/j.molcel.2016.02.018 (2016).
29 Rodrigues, C. H., Pires, D. E. & Ascher, D. B. DynaMut: predicting the impact of mutations on protein conformation, flexibility and stability. Nucleic Acids Res 46, W350-W355, doi:10.1093/nar/gky300 (2018).
30 Xie, H. et al. High-fidelity SaCas9 identified by directional screening in human cells. PLoS Biol 18, e3000747-e3000747, doi:10.1371/journal.pbio.3000747 (2020).
31 Kiani, S. et al. Cas9 gRNA engineering for genome editing, activation and repression. Nat Methods 12, 1051-1054, doi:10.1038/nmeth.3580 (2015).
32 Matharu, N. et al. CRISPR-mediated activation of a promoter or enhancer rescues obesity caused by haploinsufficiency. Science 363, doi:10.1126/science.aau0629 (2019).
33 Huang, T. P. et al. Circularly permuted and PAM-modified Cas9 variants broaden the targeting scope of base editors. Nat Biotechnol 37, 626-631, doi:10.1038/s41587-019-0134-y (2019).
34 Richter, M. F. et al. Phage-assisted evolution of an adenine base editor with improved Cas domain compatibility and activity. Nat Biotechnol 38, 883-891, doi:10.1038/s41587-020-0453-z (2020).
35 Liu, P. et al. Improved prime editors enable pathogenic allele correction and cancer modelling in adult mice. Nat Commun 12, 2121, doi:10.1038/s41467-021-22295-w (2021).
36 Gao, W., Mahajan, S. P., Sulam, J. & Gray, J. J. Deep Learning in Protein Structural Modeling and Design. Patterns (NY) 1, 100142, doi:10.1016/j.patter.2020.100142 (2020).
37 Rodrigues, C. H., Pires, D. E. & Ascher, D. B. DynaMut: predicting the impact of mutations on protein conformation, flexibility and stability. Nucleic Acids Res 46, W350-W355, doi:10.1093/nar/gky300 (2018).
38 Kellogg, E. H., Leaver-Fay, A. & Baker, D. Role of conformational sampling in computing mutation-induced changes in protein structure and stability. Proteins 79, 830-838, doi:10.1002/prot.22921 (2011).
39 Chaudhury, S., Lyskov, S. & Gray, J. J. PyRosetta: a script-based interface for implementing molecular modeling algorithms using Rosetta. Bioinformatics 26, 689-691, doi:10.1093/bioinformatics/btq007 (2010).
40 Sun, M. G., Seo, M. H., Nim, S., Corbi-Verge, C. & Kim, P. M. Protein engineering by highly parallel screening of computationally designed variants. Sci Adv 2, e1600692, doi:10.1126/sciadv.1600692 (2016).
41 Wan, Y. K., Choi, G. C. G. & Wong, A. S. L. High-Throughput Protein Engineering by Massively Parallel Combinatorial Mutagenesis. Methods Mol Biol 2199, 3-12, doi:10.1007/978-1-0716-0892-0_1 (2021).
42 Sarfati, H., Naftaly, S., Papo, N. & Keasar, C. Predicting mutant outcome by combining deep mutational scanning and machine learning. Proteins, doi:10.1002/prot.26184 (2021).
43 Guschin, D. Y. et al. A rapid and general assay for monitoring endogenous gene modification. Methods Mol Biol 649, 247-256, doi:10.1007/978-1-60761-753-2_15 (2010).
44 Tsai, S. Q., Topkar, V. V., Joung, J. K. & Aryee, M. J. Open-source guideseq software for analysis of GUIDE-seq data. Nat Biotechnol 34, 483, doi:10.1038/nbt.3534 (2016).
45 Wu, Y. et al. Highly efficient therapeutic gene editing of human hematopoietic stem cells. Nat Med 25, 776-783, doi:10.1038/s41591-019-0401-y (2019).
46 Fu, Y., Sander, J. D., Reyon, D., Cascio, V. M. & Joung, J. K. Improving CRISPR-Cas nuclease specificity using truncated guide RNAs. Nat Biotechnol 32, 279-284, doi:10.1038/nbt.2808 (2014).

We claim:

1. A Cas9 protein comprising SEQ ID NOs: 3 or 4 with an amino acid mutation at residues 888, 889, or a combination thereof of and/or at residues 988, 989, or a combination thereof.

2. The Cas9 protein of claim 1, comprising SEQ ID NO: 40, wherein the mutation at residue 888 is N to Q.

3. The Cas9 protein of claim 1, comprising SEQ ID NO: 41, wherein the mutation at residue 888 is N to Q and at residue 889 is A to S.

4. The Cas9 protein of claim 1, comprising SEQ ID NO: 42, wherein the mutation at residue 888 is N to H and at residue 889 is A to Q.

5. The Cas9 protein of claim 1, comprising SEQ ID NO: 43, wherein the mutation at residue 888 is N to S and at residue 889 is A to Q.

6. The Cas9 protein of claim 1, comprising SEQ ID NO: 44, wherein the mutation at residue 888 is N to R and at residue 889 is A to Q.

7. The Cas9 protein of claim 1, comprising SEQ ID NO: 50, wherein the mutation at residue 888 is N to G.

8. A method of enhancing the activity of KKH-SaCas9, the method comprising:
mutating residue N888, residue A889, or a combination thereof of KKH-SaCas9.

9. The method of claim 8, wherein KKH-SaCas9 comprises SEQ ID NO: 3 or 4.

10. The method of claim 8, wherein the mutation at residue 888 is N to Q.

11. The method of claim 8, wherein the mutation at residue 888 is N to Q and at residue 889 is A to S.

12. The method of claim 8, wherein the mutation at residue 888 is N to H and at residue 889 is A to Q.

13. The method of claim 8, wherein the mutation at residue 888 is N to S and at residue 889 is A to Q.

14. The method of claim 8, wherein the mutation at residue 888 is N to R and at residue 889 is A to Q.

15. The method of claim 8, wherein the mutation at residue 888 is N to G.

16. A method of enhancing the activity of KKH-SaCas9, the method comprising:
mutating KKH-SaCas9 at positions N986, D987, L988, L989, or any combination thereof.

17. The method of claim 16, wherein KKH-SaCas9 comprises SEQ ID NO: 3 or 4.

18. A method of machine learning-based in silico screens for genome editing protein engineering, comprising:
populating a predictive machine learning model with an input dataset comprising empirical measurements of on-target activities of sgRNAs paired with a screening library of genome editing enzyme variants;
running the predictive machine learning model with predefined parameters; and
evaluating performance of the predictive machine learning model.

19. The method according to claim 18, wherein enrichment scores of the empirical measurements are min-max normalized to scaled fitness scores ranging between 0 and 1.

20. The method according to claim 18, wherein the input dataset includes empirical measurements of different percentages generated to test minimal number of inputs for effective selection of top variants by predictions of the machine learning model.

21. The method according to claim 18, the populating a predictive machine learning model with an input dataset further comprising generating a plurality of replicates of the input dataset based on a randomized selection scheme or a diverse selection scheme for variants.

22. The method according to claim 21, wherein the generating a plurality of replicates based on the randomized selection scheme comprises randomly selecting a pre-defined number of enrichment scores.

23. The method according to claim 21, wherein the generating a plurality of replicates based on the diverse selection scheme comprises keeping running randomly sampling variants with available enrichment scores until no variants sharing more than p 1-mismatch-neighbours and q 2-mismatches neighbours are present in the input dataset.

24. The method according to claim 18, wherein the predefined parameters comprise Belper and Georgiev embeddings of full-length amino-acid sequences of SpCas9 (UniProtKB—Q99ZW2 (CAS9_STRP1)) and SaCas9 (UniProtKB—J7RUA5 (CAS9_STAAU)) substituted with designated variant's amino-acid residue combination.

25. The method according to claim 18, wherein the performance of the predictive machine learning model includes precision, specificity, and sensitivity of the embeddings of the predictive machine learning model.

26. The method according to claim 18, wherein the evaluating performance of the predictive machine learning model comprises counting numbers of true positives, true negatives, false positives, and false negatives for each result and deriving metrics of the performance of the predictive machine learning model based on the numbers counted.

27. A method combining machine learning-based in silico screens for genome editing protein engineering with downstream structure-guided rational design, comprising:
populating a predictive machine learning model with an input dataset comprising empirical measurements of on-target activities of sgRNAs paired with a screening library of genome editing enzyme variants;
running the predictive machine learning model with predefined parameters;
evaluating performance of the predictive machine learning model;
constructing a plasmid;
cell culturing and transducing;
conducting fluorescent protein disruption assays;
performing immunoblot analysis;
performing a T7 endonuclease I assay;
performing GUIDE-seq; and
performing molecular dynamic simulations on the variants.

28. A computer program product, comprising:
a non-transitory computer-executable storage device having computer readable program instructions embodied thereon that when executed by a computer cause the computer to perform machine learning-based in silico screens for genome editing protein engineering, the computer-executable program instruction comprising:
populating a predictive machine learning model with an input dataset comprising empirical measurements of on-target activities of sgRNAs paired with a screening library of genome editing enzyme variants;
running the predictive machine learning model with predefined parameters; and
evaluating performance of the predictive machine learning model.

29. The computer program product according to claim 28, wherein enrichment scores of the empirical measurements are min-max normalized to scaled fitness scores ranging between 0 and 1.

30. The computer program product according to claim 28, wherein the input dataset includes empirical measurements of different percentages generated to test minimal number of inputs for effective selection of top variants by predictions of the machine learning model.

31. The computer program product according to claim 28, the populating a predictive machine learning model with an input dataset further comprising generating a plurality of replicates of the input dataset based on a randomized selection scheme or a diverse selection scheme for variants.

32. The computer program product according to claim 31, wherein the generating a plurality of replicates based on the randomized selection scheme comprises randomly selecting a pre-defined number of enrichment scores.

33. The computer program product according to claim 31, wherein the generating a plurality of replicates based on the diverse selection scheme comprises keeping running randomly sampling variants with available enrichment scores until no variants sharing more than p 1-mismatch-neighbours and q 2-mismatches neighbors are present in the input dataset.

34. The computer program product according to claim 28, wherein the predefined parameters comprise Belper and Georgiev embeddings of full-length amino-acid sequences of SpCas9 (UniProtKB—Q99ZW2 (CAS9_STRP1)) and SaCas9 (UniProtKB—J7RUA5 (CAS9_STAAU)) substituted with designated variant's amino-acid residue combination.

35. The computer program product according to claim 28, wherein the performance of the predictive machine learning model includes precision, specificity, and sensitivity of the embeddings of the predictive machine learning model.

36. The computer program product according to claim 28, wherein the evaluating performance of the predictive machine learning model comprises counting numbers of true positives, true negatives, false positives, and false negatives for each result and deriving metrics of the performance of the predictive machine learning model based on the numbers counted.

37. The method according to claim 27, wherein the plasmid is obtained by polymerase chain reaction (PCR), restriction enzyme digestion, ligation, one-pot ligation, or Gibson assembly.
